(19) United States
(12) Patent Application Publication
(10) Pub. No.: US 2023/0226002 A1
(43) Pub. Date: Jul. 20, 2023

(54) RETINAL BIOAVAILBILITY OF SYNTHETIC VERY-LONG-CHAIN POLYUNSATURATED FATTY ACIDS

(71) Applicant: University of Utah Research Foundation, Salt Lake City, UT (US)

(72) Inventors: Aruna Gorusupudi, Midvale, UT (US); Paul S. Bernstein, Salt Lake City, UT (US); Jon D. Rainier, Salt Lake City, UT (US); Rameshu Rallabandi, Salt Lake City, UT (US)

(21) Appl. No.: 18/009,973

(22) PCT Filed: Jun. 15, 2021
(86) PCT No.: PCT/US2021/037524

§ 371 Date: Dec. 12, 2022

Related U.S. Application Data
(60) Provisional application No. 63/039,331, filed on Jun. 15, 2020.

(51) Int. Cl. A61K 31/202; A61K 9/127; A61P 27/02; A61K 47/22

(52) U.S. Cl. CPC A61K 31/202; A61K 9/127; A61P 27/02; A61K 47/22

(57) ABSTRACT

The rare non-dietary very-long-chain polyunsaturated fatty acids (VLC-PUFAs) uniquely found in retina and a few other tissues play a clinically significant role in retinal degeneration and development, but their physiological and interventional research has been hampered by scarcity of pure VLC-PUFAs. Disclosed herein are methods of making fatty acids, including VLC-PUFAs, and methods of using these fatty acids in, for example, treating eye disorders and supplementing the diet of a female subject who is pregnant, desiring to become pregnant, or lactating. Also disclosed are compositions containing fatty acids and methods of making and using same. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/039,331, filed on Jun. 15, 2020, the contents of which are hereby incorporated by reference in their entirety.

### BACKGROUND

Very long-chain polyunsaturated fatty acids (VLC-PUFAs) are a special class of non-dietary fatty acids (C≥24) that cannot be synthesized de novo in vertebrates and are rarely consumed in normal diets. They have a unique hybrid structure with a proximal end more characteristic of typical saturated fatty acids and a distal end more characteristic of common PUFAs like docosahexaenoic acid (DHA). It is believed that these properties enhance membrane fluidity and help maintain the highly curved membrane disks of the photoreceptor outer segments, which makes VLC-PUFAs of particular interest and importance. VLC-PUFAs are synthesized in vivo in retina from specific precursors, such as eicosapentaenoic acid (EPA) and arachidonic acid (AA), through the action of an enzyme known as ELOVL4. Genetic defects in ELOVL4 result in dominant Stargardt disease (STGD3) (Bernstein, et al. (2001) Invest. Ophthalmol. Vis. Sci. 42: 3331-6; Zhang, et al. (2001) Nat. Genet. 27: 89-93), an early-onset macular dystrophy, analogous to the more common disorder, dry age-related macular degeneration (dAMD). It was recently observed that donor retinal punches with dAMD (Gorusupudi, et al. (2016) J. Lipid. Res. 57: 499-508) and diabetic retinopathy (DR) (Gorusupudi, et al. (2018) Mol. Nutr. Food Res. 63: e1801058) have lower levels of VLC-PUFAs, and associated ELOVL4 dysfunction (Kady, et al. (2018) Diabetes 67: 769-781).

Clinical studies also suggest that VLC-PUFA supplementation could be a potential treatment for STGD3 (Hubbard, et al. (2006) Arch, Ophthalmol. 124: 257-63; Choi, et al. (2018) Ophthalmic Genet. 39: 307-313), macular dystrophies (Gorusupudi, et al. (2016) J. Lipid. Res. 57: 499-508), and diabetic retinopathies (Gorusupudi, et al. (2018) Mol. Nutr. Food Res. 63: e1801058), but a major limiting factor is the lack of availability of pure VLC-PUFAs that has precluded laboratory animal and clinical studies. Thus, there remains a need for VLC-PUFAs, compositions containing VLC-PUFAs, and methods of making and using same.

### SUMMARY

In accordance with the purpose(s) of the invention, as embodied and broadly described herein, the invention, in one aspect, relates to polyunsaturated fatty acids and compositions containing polyunsaturated fatty acids for use in the prevention and treatment of eye disorders such as, for example, Stargardt-3 (STGD3) disease, macular dystrophy, age-related macular degeneration (AMD), and diabetic retinopathy, and for use in supplementing a female subject's diet, wherein the female subject is pregnant, desiring to become pregnant, as further described herein, or lactating. The invention also relates to methods of making fatty acids such as, for example, polyunsaturated fatty acids.

Thus, disclosed are compositions comprising: (a) a liposome; (b) vitamin E; and (c) a polyunsaturated fatty acid having a chain length of at least 24 carbon atoms, or a pharmaceutically acceptable salt thereof.

Also disclosed are methods for treating an eye disorder in a subject in need thereof, the method comprising administering to the subject an effective amount of a disclosed composition.

Also disclosed are methods for supplementing a female subject's diet, the method comprising administering to the female subject an effective amount of a disclosed composition, wherein the female subject is pregnant, desiring to become pregnant, or lactating.

Also disclosed are kits comprising a disclosed composition, and one or more of: (a) an agent known for treating an eye disorder; (b) an agent known for supplementing a female subject's diet, wherein the female subject is pregnant, desiring to become pregnant, or lactating; (c) instructions for administering the composition in connection with treating an eye disorder or supplementing a female subject's diet, wherein the female subject is pregnant, desiring to become pregnant, or lactating; and (d) instructions for treating an eye disorder or supplementing a female subject's diet, wherein the female subject is pregnant, desiring to become pregnant, or lactating.

Also disclosed are methods for making a fatty acid having a chain length of at least 18 carbon atoms, the method comprising coupling, in the absence of heavy metals, (a) an activated alkyl halide having a protected alcohol and a linear chain of at least two carbon atoms with (b) an aldehyde having at least 16 carbon atoms, thereby providing the fatty acid. In a further aspect, the fatty acid is a polyunsaturated fatty acid and the aldehyde has from four to eight cis carbon-carbon double bonds.

Also disclosed are fatty acids produced by a disclosed method. In a further aspect, the fatty acid is a polyunsaturated fatty acid having from four to eight cis carbon-carbon double bonds.

Also disclosed are compositions comprising a polyunsaturated fatty acid produced by a disclosed method and a liposome.

Also disclosed are methods for treating an eye disorder in a subject in need thereof, the method comprising administering to the subject an effective amount of a polyunsaturated fatty acid produced by a disclosed method.

Also disclosed are methods for supplementing a female subject's diet, the method comprising administering to the female subject an effective amount of a polyunsaturated fatty acid produced by a disclosed method, wherein the female subject is pregnant, desiring to become pregnant, or lactating.

Also disclosed are kits comprising a polyunsaturated fatty acid produced by a disclosed method, and one or more of: (a) an agent known for treating of an eye disorder; (b) an agent know for supplementing a female subject's diet, wherein the female subject is pregnant, desiring to become pregnant, or lactating; (c) instructions for administering the composition in connection with treating an eye disorder or supplementing a female subject's diet, wherein the female subject is pregnant, desiring to become pregnant, or lactating; and (d) instructions for treating an eye disorder or supplementing a female subject's diet wherein the female subject is pregnant, desiring to become pregnant, or lactating.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative schematic illustrating a synthetic approach to VLC-PUFA 32:6 n-3.

FIG. 2A and FIG. 2B show representative data illustrating the biophysical effects of VLC-PUFA 32:6 n-3 on model membranes.

FIG. 3A-F show representative data illustrating the bioavailability and functional effects of VLC-PUA 32:6 n-3.

FIG. 4A-I show representative schematics illustrating synthesis of VLC-PUFAs.

FIG. 5 shows representative data illustrating the % of total VLC-PUFA in serum (left panel) and retina (right panel) of wild mice after administration of 500 mg/kg/day of VLC-PUFA in safflower oil (100 μl).

FIG. 6 shows a representative schematic illustrating the synthesis of a deuterated VLC-PUFA.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present polyunsaturated fatty acids, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the fatty acids, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific fatty acid employed; the duration of the treatment; drugs used in combination or coincidental with the specific fatty acid employed and like factors well known in the medical arts. For example, it is well within the skill of the art to start doses of a fatty acid at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed fatty acid, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed fatty acid, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂¹A′, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘)}₂; SiR^{∘}₃; —(C₁₋₄ straight or branched alkylene)O—N(R^{∘})₂; or —(C₁₋₄ straight or branched alkylene)C(O)O—N(R^{∘)}₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, −OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R¹, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

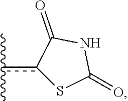

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

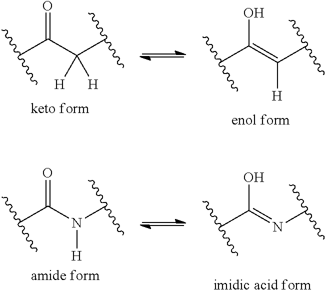 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

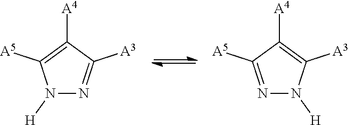

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

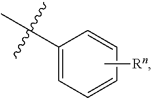

which is understood to be equivalent to a formula:

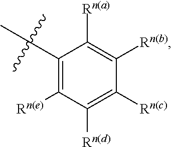

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, Mass.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Methods for Making a Fatty Acid

In one aspect, the invention relates to methods of making fatty acids, such as, for example, polyunsaturated fatty acids, useful in treating eye disorders (e.g., Stargardt-3 (STGD3) disease, macular dystrophy, age-related macular degeneration (AMD), diabetic retinopathy) and in supplementing a female subject's diet, wherein the female subject is pregnant, desiring to become pregnant, or lactating.

Thus, in one aspect, disclosed are methods for making a fatty acid having a chain length of at least 18 carbon atoms, the method comprising coupling, in the absence of heavy metals, (a) an activated alkyl halide having a protected alcohol and a linear chain of at least two carbon atoms with (b) an aldehyde having at least 16 carbon atoms, thereby providing the fatty acid. In a further aspect, the aldehyde has from four to eight cis carbon-carbon double bonds, and wherein the fatty acid is a polyunsaturated fatty acid.

In various aspects, the method further comprises activating an alkyl halide, thereby providing the activated alkyl halide. In a further aspect, the alkyl halide is activated via reaction with magnesium metal.

In various aspects, the method further comprises reducing an alcohol after the coupling step.

In various aspects, the method further comprises deprotecting the protected alcohol.

In various aspects, the method further comprises oxidizing the unprotected alcohol.

In various aspects, the coupling is in the absence of heavy metals. In various further aspects, the method is in the absence of heavy metals. Examples of heavy metals include, but are not limited to, mercury, cadmium, arsenic, chromium, thallium, and lead.

In various aspects, the fatty acid is a polyunsaturated fatty acid having a structure represented by a formula:

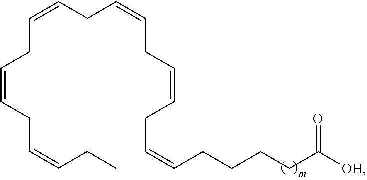

wherein m is 2, 3, 4, 5, 6, 7, 8, or 9. In a further aspect, m is 2, 3, 4, 5, 6, 7, or 8. In a still further aspect, m is 2, 3, 4, 5, 6, or 7. In yet a further aspect, m is 2, 3, 4, 5, or 6. In an even further aspect, m is 2, 3, 4, or 5. In a still further aspect, m is 2, 3, or 4. In yet a further aspect, m is 2 or 3.

In various aspects, the fatty acid is a polyunsaturated fatty acid having a structure represented by a formula:

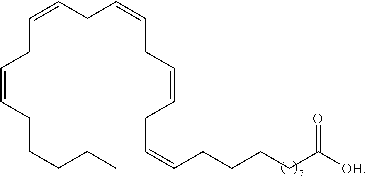

In various aspects, the fatty acid is a polyunsaturated fatty acid having a structure represented by a formula:

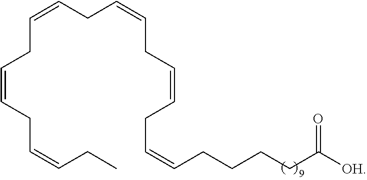

In various aspects, the fatty acid is a polyunsaturated fatty acid having a structure represented by a formula:

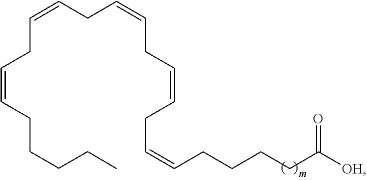

wherein m is 2, 3, 4, 5, 6, 7, 8, or 9. In a further aspect, m is 2, 3, 4, 5, 6, 7, or 8. In a still further aspect, m is 2, 3, 4, 5, 6, or 7. In yet a further aspect, m is 2, 3, 4, 5, or 6. In an even further aspect, m is 2, 3, 4, or 5. In a still further aspect, m is 2, 3, or 4. In yet a further aspect, m is 2 or 3.

In various aspects, the fatty acid is a polyunsaturated fatty acid having a structure represented by a formula:

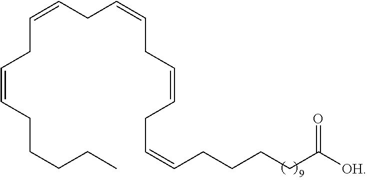

In various aspects, the method comprises: (a) activating an alkyl halide having a protected alcohol and a linear chain of at least two carbon atoms; (b) coupling, in the absence of heavy metals, the activated alkyl halide and an aldehyde having at least 16 carbon atoms and of from four to eight cis carbon-carbon double bonds; (c) deprotecting the protected alcohol; and (d) oxidizing the unprotected alcohol. In various further aspects, the method is in the absence of heavy metals.

In various aspects, the fatty acid is a polyunsaturated fatty acid, and wherein the method comprises: (a) activating an alkyl halide having a structure:

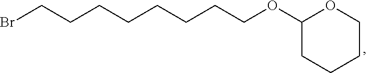 [SMILES: BrCCCCCCCCOC1CCCCO1]

via reaction with magnesium metal, thereby providing a Grignard reagent; (b) coupling, in the absence of heavy metals, the Grignard reagent with an aldehyde having a structure:

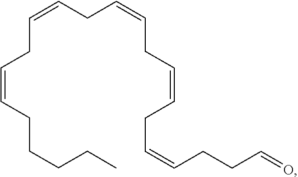 [SMILES: CCCCC/C=C\C/C=C\C/C=C\C/C=C\C/C=C\CCC=O]

thereby providing a secondary alcohol having a structure:

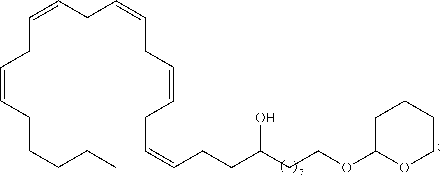

(c) reducing the secondary alcohol; (d) deprotecting the protected alcohol; and (e) oxidizing the unprotected alcohol, thereby providing a polyunsaturated fatty acid having a structure:

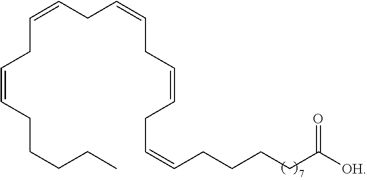

In a further aspect, the method is in the absence of heavy metals.

In various aspects, the fatty acid is a polyunsaturated fatty acid, and wherein the method comprises: (a) activating an alkyl halide having a structure:

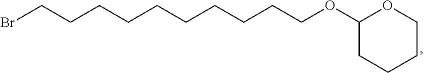 [SMILES: BrCCCCCCCCCCOC1CCCCO1]

via reaction with magnesium metal, thereby providing a Grignard reagent; (b) coupling, in the absence of heavy metals, the Grignard reagent with an aldehyde having a structure:

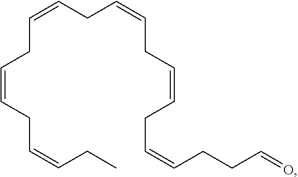 [SMILES: CC/C=C\C/C=C\C/C=C\C/C=C\C/C=C\C/C=C\CCC=O]

thereby providing a secondary alcohol having a structure:

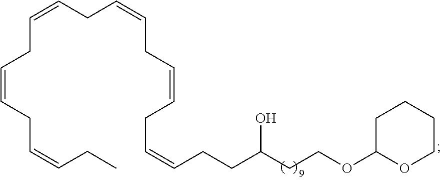

(c) reducing the secondary alcohol; (d) deprotecting the protected alcohol; and (e) oxidizing the unprotected alcohol, thereby providing a polyunsaturated fatty acid having a structure:

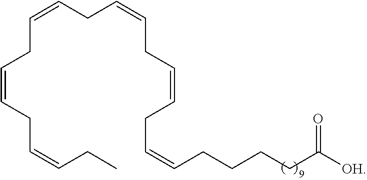

In a further aspect, the method is in the absence of heavy metals.

In various aspects, the fatty acid is a polyunsaturated fatty acid, and wherein the method comprises: (a) activating an alkyl halide having a structure:

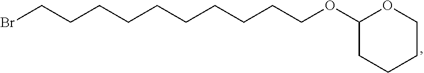 [SMILES: BrCCCCCCCCCCOC1CCCCO1]

via reaction with magnesium metal, thereby providing a Grignard reagent; (b) coupling, in the absence of heavy metals, the Grignard reagent with an aldehyde having a structure:

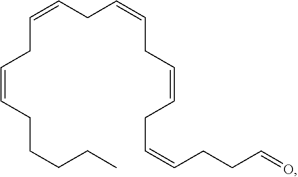 [SMILES: CCCCC/C=C\C/C=C\C/C=C\C/C=C\C/C=C\CCC=O]

thereby providing a secondary alcohol having a structure:

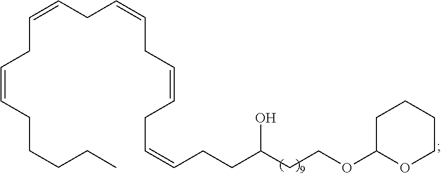

(c) reducing the secondary alcohol; (d) deprotecting the protected alcohol; and (e) oxidizing the unprotected alcohol, thereby providing a polyunsaturated fatty acid having a structure:

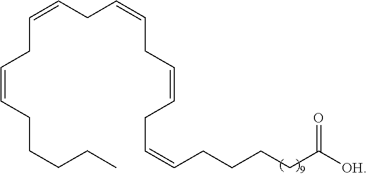

In a further aspect, the method is in the absence of heavy metals.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Activated Alkyl Halides

In one aspect, the disclosed method comprises coupling an activated alkyl halide having a protected alcohol and a linear chain of at least two carbon atoms.

In various aspects, the activated alkyl halide has a linear chain of at least two carbon atoms, at least three carbon atoms, at least four carbon atoms, at least five carbon atoms, at least six carbon atoms, at least seven carbon atoms, at least eight carbon atoms, or at least nine carbon atoms. In a further aspect, the activated alkyl halide has a linear chain of from two carbon atoms to fifteen carbon atoms, from four carbon atoms to fifteen carbon atoms, from six carbon atoms to fifteen carbon atoms, from eight carbon atoms to fifteen carbon atoms, from ten carbon atoms to fifteen carbon atoms, from two carbon atoms to thirteen carbon atoms, from two carbon atoms to eleven carbon atoms, from two carbon atoms to nine carbon atoms, from two carbon atoms to seven carbon atoms, from four carbon atoms to thirteen carbon atoms, or from six carbon atoms to eleven carbon atoms. In a still further aspect, the activated alkyl halide has a linear chain of nine or ten carbon atoms.

In various aspects, the activated alkyl halide comprises a structure selected from:

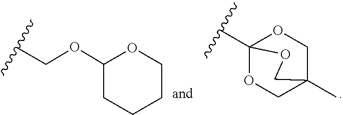

In various aspects, the activated alkyl halide comprises a structure

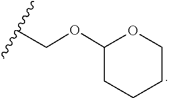 [SMILES: *COC1CCCCO1]

In various aspects, the activated alkyl halide comprises a structure

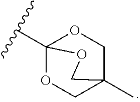 [SMILES: *C12OCC(C)(CO1)CO2]

In various aspects, the method further comprises activating an alkyl halide, thereby providing the activated alkyl halide. In a further aspect, the alkyl halide has a linear chain of at least two carbon atoms, at least three carbon atoms, at least four carbon atoms, at least five carbon atoms, at least six carbon atoms, at least seven carbon atoms, at least eight carbon atoms, or at least nine carbon atoms. In a further aspect, the alkyl halide has a linear chain of from two carbon atoms to fifteen carbon atoms, from four carbon atoms to fifteen carbon atoms, from six carbon atoms to fifteen carbon atoms, from eight carbon atoms to fifteen carbon atoms, from ten carbon atoms to fifteen carbon atoms, from two carbon atoms to thirteen carbon atoms, from two carbon atoms to eleven carbon atoms, from two carbon atoms to nine carbon atoms, from two carbon atoms to seven carbon atoms, from four carbon atoms to thirteen carbon atoms, or from six carbon atoms to eleven carbon atoms. In a still further aspect, the alkyl halide has a linear chain of nine or ten carbon atoms.

In a further aspect, the alkyl halide has a structure represented by a formula:

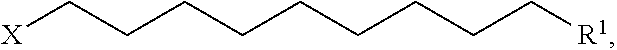

wherein X is a halogen; and wherein R¹ is a protected alcohol (i.e., an alcohol having a protecting group that, upon removal, yields an alcohol). Examples of protecting groups include, but are not limited to, acetyl, benzoyl, benzyl, β-methoxyethoxymethyl ether, dimethoxytrityl, methoxymethyl ether, p-methoxybenzyl ether, pivaloyl, tetrahydropyranyl, tetrahydrofuran, trityl, silyl ether protecting groups (e.g., trimethylsilyl, tert-butyldimethylsilyl, triisopropylsilyloxymethyl, triisopropylsilyl), and methyl ethers.

In various aspects, X is selected from —F, —Br, or —Cl. In a further aspect, X is selected from —Br and —Cl. In a still further aspect, X is —Br.

In various aspects, the alkyl halide has a structure selected from:

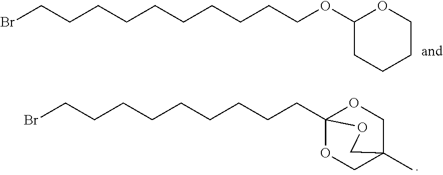

In various aspects, alkyl halide has a structure:

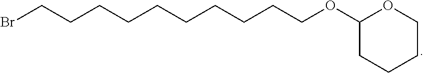 [SMILES: BrCCCCCCCCCCOC1CCCCO1]

In various aspects, alkyl halide has a structure:

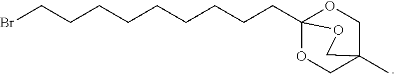 [SMILES: CC12COC(CCCCCCCCCBr)(OC1)OC2]

In various aspects, the alkyl halide is activated via reaction with magnesium metal.

In various aspects, the activated alkyl halide is a Grignard reagent such as, for example, a magnesium-based Grignard reagent.

2. Aldehydes

In one aspect, the disclosed method comprises coupling an aldehyde having at least 16 carbon atoms. In a further aspect, the aldehyde has from four to eight cis carbon-carbon double bonds.

Thus, in various aspects, the aldehyde has a linear chain of at least 16 carbon atoms, at least 18 carbon atoms, at least 20 carbon atoms, or at least 22 carbon atoms. In various aspects, the aldehyde has a linear chain of from about 16 carbon atoms to about 26 carbon atoms, from about 18 carbon atoms to about 26 carbon atoms, from about 20 carbon atoms to about 26 carbon atoms, from about 22 carbon atoms to about 26 carbon atoms, from about 24 carbon atoms to about 26 carbon atoms, from about 16 carbon atoms to about 24 carbon atoms, from about 16 carbon atoms to about 22 carbon atoms, from about 16 carbon atoms to about 20 carbon atoms, from about 16 carbon atoms to about 18 carbon atoms, from about 18 carbon atoms to about 24 carbon atoms, or from about 20 carbon atoms to about 24 carbon atoms.

In various aspects, the aldehyde has from four to eight cis carbon-carbon double bonds, from four to seven cis carbon-carbon double bonds, from four to six cis carbon-carbon double bonds, from five to eight cis carbon-carbon double bonds, from six to eight cis carbon-carbon double bonds, or from five to seven cis carbon-carbon double bonds.

In various aspects, the aldehyde is formed by reducing a carboxylic acid or an ester, thereby providing an alcohol, followed by oxidation.

In various aspects, the aldehyde has a structure represented by a formula:

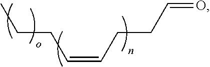

wherein n is 4, 5, 6, 7, or 8; and wherein o is 0, 1, 2, or 3.

Thus, in various aspects, n is selected from 4, 5, 6, 7, and 8. In a further aspect, n is selected from 5, 6, 7, and 8. In a still further aspect, n is selected from 6, 7, and 8. In yet a further aspect, n is selected from 7 and 8.

In various aspects, o is selected from 0, 1, 2, and 3. In a further aspect, o is selected from 0, 1, and 2. In a still further aspect, o is selected from 0 and 1.

In various aspects, the aldehyde has a structure represented by a formula:

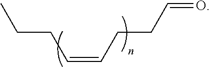

In various aspects, the aldehyde has a structure:

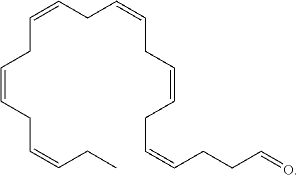 [SMILES: CC/C=C\C/C=C\C/C=C\C/C=C\C/C=C\C/C=C\CCC=O]

In various aspects, the aldehyde has a structure represented by a formula:

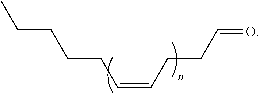

In various aspects, the aldehyde has a structure:

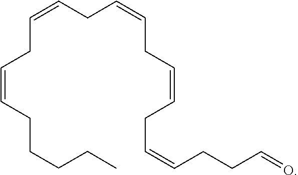 [SMILES: CCCCC/C=C\C/C=C\C/C=C\C/C=C\C/C=C\CCC=O]

3. Fatty Acids

In one aspect, disclosed are fatty acids produced by a disclosed method. Thus, in various aspects, the fatty acid is produced by coupling, in the absence of heavy metals, (a) an activated alkyl halide having a protected alcohol and a linear chain of at least two carbon atoms with (b) an aldehyde having at least 16 carbon atoms, thereby providing the fatty acid. In a further aspect, the fatty acid has a chain length of at least 18 carbon atoms. In a still further aspect, the fatty acid is a polyunsaturated fatty acid.

In one aspect, the disclosed method provides a fatty acid such as, for example, a polyunsaturated fatty acid. In various aspects, the polyunsaturated fatty acid has a chain length of at least 24 carbon atoms, a chain length of at least 26 carbon atoms, a chain length of at least 28 carbon atoms, a chain length of at least 30 carbon atoms, a chain length of at least 32 carbon atoms, or a chain length of at least 34 carbon atoms. In a further aspect, the polyunsaturated fatty acid has a chain length of from 24 carbon atoms to 36 carbon atoms, from 26 carbon atoms to 36 carbon atoms, from 28 carbon atoms to 36 carbon atoms, from 30 carbon atoms to 36 carbon atoms, from 32 carbon atoms to 36 carbon atoms, from 34 carbon atoms to 36 carbon atoms, from 26 carbon atoms to 34 carbon atoms, from 28 carbon atoms to 34 carbon atoms, from 30 carbon atoms to 34 carbon atoms, or from 32 carbon atoms to 34 carbon atoms.

In various aspects, the disclosed method provides a plurality of polyunsaturated fatty acids having a chain length of at least 24 carbon atoms. In various further aspects, the plurality of polyunsaturated fatty acids have different chain lengths. Thus, in various aspects, each chain length is from 24 carbon atoms to 36 carbon atoms, from 26 carbon atoms to 36 carbon atoms, from 28 carbon atoms to 36 carbon atoms, from 30 carbon atoms to 36 carbon atoms, from 32 carbon atoms to 36 carbon atoms, from 34 carbon atoms to 36 carbon atoms, from 26 carbon atoms to 34 carbon atoms, from 28 carbon atoms to 34 carbon atoms, from 30 carbon atoms to 34 carbon atoms, or from 32 carbon atoms to 34 carbon atoms.

In various aspects, the polyunsaturated fatty acid is an n-3 polyunsaturated fatty acid. For example, the n-3 polyunsaturated fatty acid is selected from a 3n3, 4n3, 5n3, 6n3, 7n3, and 8n3 fatty acid. In a further aspect, the n-3 polyunsaturated fatty acid is a 6n3.

In various aspects, the polyunsaturated fatty acid has a chain length of from 24 carbon atoms to 36 carbon atoms, and is an n-3 polyunsaturated fatty acid. In a further aspect, the polyunsaturated fatty acid has a chain length of from 26 carbon atoms to 36 carbon atoms, and is an n-3 polyunsaturated fatty acid. In a still further aspect, the polyunsaturated fatty acid has a chain length of from 28 carbon atoms to 36 carbon atoms, and is an n-3 polyunsaturated fatty acid. In yet a further aspect, the polyunsaturated fatty acid has a chain length of from 28 carbon atoms to 34 carbon atoms, and is an n-3 polyunsaturated fatty acid.

In various aspects, the polyunsaturated fatty acid has a chain length of 28 carbon atoms, and is an n-3 polyunsaturated fatty acid. In a further aspect, the polyunsaturated fatty acid has a chain length of 30 carbon atoms, and is an n-3 polyunsaturated fatty acid. In a still further aspect, the polyunsaturated fatty acid has a chain length of 32 carbon atoms, and is an n-3 polyunsaturated fatty acid. In yet a further aspect, the polyunsaturated fatty acid has a chain length of 34 carbon atoms, and is an n-3 polyunsaturated fatty acid. In an even further aspect, the polyunsaturated fatty acid has a chain length of 36 carbon atoms, and is an n-3 polyunsaturated fatty acid.

In various aspects, the polyunsaturated fatty acid is an n6 polyunsaturated fatty acid. For example, the n6 polyunsaturated fatty acid is selected from a 3n6, 4n6, 5n6, 6n6, and 7n6 fatty acid.

In various aspects, the polyunsaturated fatty acid has a chain length of from 24 carbon atoms to 36 carbon atoms, and is an n6 polyunsaturated fatty acid. In a further aspect, the polyunsaturated fatty acid has a chain length of from 26 carbon atoms to 36 carbon atoms, and is an n6 polyunsaturated fatty acid. In a still further aspect, the polyunsaturated fatty acid has a chain length of from 28 carbon atoms to 36 carbon atoms, and is an n6 polyunsaturated fatty acid. In yet a further aspect, the polyunsaturated fatty acid has a chain length of from 28 carbon atoms to 34 carbon atoms, and is an n6 polyunsaturated fatty acid.

In various aspects, the polyunsaturated fatty acid has a chain length of 28 carbon atoms, and is an n6 polyunsaturated fatty acid. In a further aspect, the polyunsaturated fatty acid has a chain length of 30 carbon atoms, and is an n6 polyunsaturated fatty acid. In a still further aspect, the polyunsaturated fatty acid has a chain length of 32 carbon atoms, and is an n6 polyunsaturated fatty acid. In yet a further aspect, the polyunsaturated fatty acid has a chain length of 34 carbon atoms, and is an n6 polyunsaturated fatty acid. In an even further aspect, the polyunsaturated fatty acid has a chain length of 36 carbon atoms, and is an n6 polyunsaturated fatty acid.

In various aspects, the polyunsaturated fatty acid is not modified. In a further aspect, the polyunsaturated fatty acid is fluorinated. In a still further aspect, the polyunsaturated fatty acid is isotopically labeled. In yet a further aspect, the polyunsaturated fatty acid is isotopically labeled with deuterium.

It is contemplated that one or more fatty acids can optionally be omitted from the disclosed invention.

It is understood that the disclosed fatty acids can be used in connection with the disclosed methods, compositions, kits, and uses.

It is understood that pharmaceutical acceptable derivatives of the disclosed fatty acids can be used also in connection with the disclosed methods, compositions, kits, and uses. The pharmaceutical acceptable derivatives of the fatty acids can include any suitable derivative, such as pharmaceutically acceptable salts, isomers, radiolabeled analogs, tautomers, and the like.

### C. Compositions

In one aspect, disclosed are compositions comprising a disclosed fatty acid, or a pharmaceutically acceptable salt thereof, and a liposome. In a further aspect, the fatty acid is a polyunsaturated fatty acid. Thus, in one aspect, disclosed are compositions comprising: (a) a liposome; (b) vitamin E; and (c) a polyunsaturated fatty acid having a chain length of at least 24 carbon atoms, or a pharmaceutically acceptable salt thereof.

In one aspect, the composition comprises a polyunsaturated fatty acid produced by a disclosed method and a liposome. Thus, in various aspects, the composition comprises a polyunsaturated fatty acid produced by coupling, in the absence of heavy metals, (a) an activated alkyl halide having a protected alcohol and a linear chain of at least two carbon atoms with (b) an aldehyde having at least 16 carbon atoms, thereby providing the polyunsaturated fatty acid. In a further aspect, the polyunsaturated fatty acid has a chain length of at least 18 carbon atoms.

In various aspects, the disclosed composition comprises a liposome. In various further aspects, the liposome is prepared from Tween-4 detergent, cholesterol, safflower oil, and/or sodium taurocholate. In a still further aspect, the liposome is a phosphatidyl choline. In yet a further aspect, the liposome is 1,2-distearoyl-sn-3-glycero-phosphocholine (DSPC).

In various aspects, the disclosed composition comprises a liposome in an amount of from about 0.5 wt % to about 1.5 wt %, about 0.5 wt % to about 1.25 wt %, about 0.5 wt % to about 1.0 wt %, about 0.75 wt % to about 1.5 wt %, about 1.0 wt % to about 1.5 wt %, or about 0.75 wt % to about 1.25 wt %, based on the total weight of the composition. In a further aspect, the disclosed composition comprises a liposome in an amount of about 0.50 wt %, about 0.75 wt %, about 1.0 wt %, about 1.25 wt %, or about 1.5 wt %, based on the total weight of the composition.

In various aspects, the disclosed composition comprises vitamin E. For example, vitamin E can be present in an amount of from about 25 mg per 100 mL liposomes to about 50 mg per 100 mL liposomes, from about 25 mg per 100 mL liposomes to about 40 mg per 100 mL liposomes, from about 25 mg per 100 mL liposomes to about 30 mg per 100 mL liposomes, from about 30 mg per 100 mL liposomes to about 50 mg per 100 mL liposomes, from about 40 mg per 100 mL liposomes to about 50 mg per 100 mL liposomes, or from about 30 mg per 100 mL liposomes to about 40 mg per 100 mL liposomes.

In various aspects, the disclosed composition comprises vitamin E in an amount of from about 0.25 wt % to about 0.75 wt %, about 0.30 wt % to about 0.75 wt %, about 0.40 wt % to about 0.75 wt %, about 0.50 wt % to about 0.75 wt %, about 0.25 wt % to about 0.70 wt %, about 0.25 wt % to about 0.60 wt %, about 0.25 wt % to about 0.50 wt %, about 0.30 wt % to about 0.70 wt %, or about 0.40 wt % to about 0.60 wt %, based on the total weight of the composition. In a further aspect, the disclosed composition comprises vitamin E in an amount of about 0.25 wt %, about 0.30 wt %, about 0.40 wt %, about 0.50 wt %, about 0.60 wt %, about 0.70 wt %, or about 0.75 wt %, based on the total weight of the composition.

In various aspects, the disclosed composition comprises a polyunsaturated fatty acid, or a pharmaceutically acceptable salt thereof. For example, the polyunsaturated fatty acid is present in an amount of from about 5 wt % to about 10 wt %, about 5 wt % to about 9 wt %, about 5 wt % to about 8 wt %, about 5 wt % to about 7 wt %, about 5 wt % to about 6 wt %, about 6 wt % to about 10 wt %, about 7 wt % to about 10 wt %, about 8 wt % to about 10 wt %, about 9 wt % to about 10 wt %, about 6 wt % to about 9 wt %, or about 7 wt % to about 8 wt %, based on the total weight of the composition.

In various aspects, the disclosed composition comprises a polyunsaturated fatty acid in an amount of from about 5 wt % to about 50 wt %, about 10 wt % to about 50 wt %, about 20 wt % to about 50 wt %, about 25 wt % to about 50 wt %, about 30 wt % to about 50 wt %, about 40 wt % to about 50 wt %, about 5 wt % to about 40 wt %, about 5 wt % to about 30 wt %, about 5 wt % to about 25 wt %, about 5 wt % to about 20 wt %, about 5 wt % to about 10 wt %, about 10 wt % to about 40 wt %, or about 20 wt % to about 30 wt %, based on the total weight of the composition.

In various aspects, the polyunsaturated fatty acid has a chain length of at least 24 carbon atoms, a chain length of at least 26 carbon atoms, a chain length of at least 28 carbon atoms, a chain length of at least 30 carbon atoms, a chain length of at least 32 carbon atoms, or a chain length of at least 34 carbon atoms. In a further aspect, the polyunsaturated fatty acid has a chain length of from 24 carbon atoms to 36 carbon atoms, from 26 carbon atoms to 36 carbon atoms, from 28 carbon atoms to 36 carbon atoms, from 30 carbon atoms to 36 carbon atoms, from 32 carbon atoms to 36 carbon atoms, from 34 carbon atoms to 36 carbon atoms, from 26 carbon atoms to 34 carbon atoms, from 28 carbon atoms to 34 carbon atoms, from 30 carbon atoms to 34 carbon atoms, or from 32 carbon atoms to 34 carbon atoms.

In various aspects, the ratio of the liposome to vitamin E to the polyunsaturated fatty acid is of from about 1:0.5:40 (wt %) to about 1:0.5:10 (wt %). In a further aspect, the ratio of the liposome to vitamin E to the polyunsaturated fatty acid is about 1:0.5:10 (wt %). In a still further aspect, the ratio of the liposome to vitamin E to the polyunsaturated fatty acid is about 1:0.5:40 (wt %).

In various aspects, the disclosed composition comprises a plurality of polyunsaturated fatty acids having a chain length of at least 24 carbon atoms. In various further aspects, the plurality of polyunsaturated fatty acids have different chain lengths. Thus, in various aspects, each chain length is from 24 carbon atoms to 36 carbon atoms, from 26 carbon atoms to 36 carbon atoms, from 28 carbon atoms to 36 carbon atoms, from 30 carbon atoms to 36 carbon atoms, from 32 carbon atoms to 36 carbon atoms, from 34 carbon atoms to 36 carbon atoms, from 26 carbon atoms to 34 carbon atoms, from 28 carbon atoms to 34 carbon atoms, from 30 carbon atoms to 34 carbon atoms, or from 32 carbon atoms to 34 carbon atoms.

In various aspects, the polyunsaturated fatty acid is an n-3 polyunsaturated fatty acid. For example, the n-3 polyunsaturated fatty acid is selected from a 3n3, 4n3, 5n3, 6n3, 7n3, and 8n3 fatty acid. In a further aspect, the n-3 polyunsaturated fatty acid is a 6n3.

In various aspects, the polyunsaturated fatty acid has a chain length of from 24 carbon atoms to 36 carbon atoms, and is an n-3 polyunsaturated fatty acid. In a further aspect, the polyunsaturated fatty acid has a chain length of from 26 carbon atoms to 36 carbon atoms, and is an n-3 polyunsaturated fatty acid. In a still further aspect, the polyunsaturated fatty acid has a chain length of from 28 carbon atoms to 36 carbon atoms, and is an n-3 polyunsaturated fatty acid. In yet a further aspect, the polyunsaturated fatty acid has a chain length of from 28 carbon atoms to 34 carbon atoms, and is an n-3 polyunsaturated fatty acid.

In various aspects, the polyunsaturated fatty acid has a chain length of 28 carbon atoms, and is an n-3 polyunsaturated fatty acid. In a further aspect, the polyunsaturated fatty acid has a chain length of 30 carbon atoms, and is an n-3 polyunsaturated fatty acid. In a still further aspect, the polyunsaturated fatty acid has a chain length of 32 carbon atoms, and is an n-3 polyunsaturated fatty acid. In yet a further aspect, the polyunsaturated fatty acid has a chain length of 34 carbon atoms, and is an n-3 polyunsaturated fatty acid. In an even further aspect, the polyunsaturated fatty acid has a chain length of 36 carbon atoms, and is an n-3 polyunsaturated fatty acid.

In various aspects, the polyunsaturated fatty acid is an n6 polyunsaturated fatty acid. For example, the n6 polyunsaturated fatty acid is selected from a 3n6, 4n6, 5n6, 6n6, and 7n6 fatty acid.

In various aspects, the polyunsaturated fatty acid has a chain length of from 24 carbon atoms to 36 carbon atoms, and is an n6 polyunsaturated fatty acid. In a further aspect, the polyunsaturated fatty acid has a chain length of from 26 carbon atoms to 36 carbon atoms, and is an n6 polyunsaturated fatty acid. In a still further aspect, the polyunsaturated fatty acid has a chain length of from 28 carbon atoms to 36 carbon atoms, and is an n6 polyunsaturated fatty acid. In yet a further aspect, the polyunsaturated fatty acid has a chain length of from 28 carbon atoms to 34 carbon atoms, and is an n6 polyunsaturated fatty acid.

In various aspects, the polyunsaturated fatty acid has a chain length of 28 carbon atoms, and is an n6 polyunsaturated fatty acid. In a further aspect, the polyunsaturated fatty acid has a chain length of 30 carbon atoms, and is an n6 polyunsaturated fatty acid. In a still further aspect, the polyunsaturated fatty acid has a chain length of 32 carbon atoms, and is an n6 polyunsaturated fatty acid. In yet a further aspect, the polyunsaturated fatty acid has a chain length of 34 carbon atoms, and is an n6 polyunsaturated fatty acid. In an even further aspect, the polyunsaturated fatty acid has a chain length of 36 carbon atoms, and is an n6 polyunsaturated fatty acid.

In various aspects, the polyunsaturated fatty acid is not modified. In a further aspect, the polyunsaturated fatty acid is fluorinated. In a still further aspect, the polyunsaturated fatty acid is isotopically labeled. In yet a further aspect, the polyunsaturated fatty acid is isotopically labeled with deuterium.

In various aspects, the fatty acids of the invention can be administered in compositions, which are formulated according to the intended method of administration. The fatty acids and compositions described herein can be formulated in a conventional manner using one or more physiologically acceptable carriers or excipients. For example, a composition can be formulated for local or systemic administration, intravenous, topical, or oral administration.

The nature of the compositions for administration is dependent on the mode of administration and can readily be determined by one of ordinary skill in the art. In various aspects, the composition is sterile or sterilizable. The compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The fatty acids featured in the invention can be administered by any standard route of administration. For example, administration can be parenteral, intravenous, subcutaneous, or oral. A fatty acid can be formulated in various ways, according to the corresponding route of administration. For example, liquid solutions can be made for administration by drops into the eye, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, Pa. 1990.

In various aspects, the disclosed compositions comprise the disclosed fatty acids (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a fatty acid or a pharmaceutically acceptable salt of the fatty acids of the invention. The fatty acids of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a fatty acid of the invention, and/or pharmaceutically acceptable salts thereof, can also be prepared in powder or liquid concentrate form.

In a further aspect, the composition is formulated for oral administration. In a still further aspect, the composition is formulated for intraventricular injection. In yet a further aspect, the composition is formulated for ocular administration.

In a further aspect, the composition comprises an effective amount of the polyunsaturated fatty acid. In a still further aspect, the effective amount is a therapeutically effective amount. In yet a further aspect, the effective amount is a prophylactically effective amount. In an even further aspect, the effective amount is a neutraceutically effective amount.

In a further aspect, the pharmaceutical composition is administered to a mammal. In a still further aspect, the mammal is a human. In an even further aspect, the human is a patient.

In a further aspect, the pharmaceutical composition is used to treat an eye disorder such as, for example, Stargardt-3 (STGD3) disease, macular dystrophy, age-related macular degeneration (AMD), and diabetic retinopathy. In a still further aspect, the pharmaceutical composition is used to supplement a female subject's diet, wherein the female subject is pregnant, desiring to become pregnant, or lactating.

It is understood that the disclosed compositions can be prepared from the disclosed fatty acids. It is also understood that the disclosed compositions can be employed in the disclosed methods of using.

### D. Methods for Treating an Eye Disorder in a Subject

In one aspect, disclosed are methods for treating an eye disorder in a subject in need thereof, the method comprising administering to the subject an effective amount of a disclosed composition. Thus, in various aspects, disclosed are methods for treating an eye disorder in a subject in need thereof, the method comprising administering to the subject an effective amount of a composition comprising: (a) a liposome; (b) vitamin E; and (c) a polyunsaturated fatty acid having a chain length of at least 24 carbon atoms, or a pharmaceutically acceptable salt thereof.

In one aspect, disclosed are methods for treating an eye disorder in a subject in need thereof, the method comprising administering to the subject an effective amount of a polyunsaturated fatty acid produced by a disclosed method. Thus, in various aspects, disclosed are methods for treating an eye disorder in a subject in need thereof, the method comprising administering to the subject an effective amount of a polyunsaturated fatty acid produced by coupling, in the absence of heavy metals, (a) an activated alkyl halide having a protected alcohol and a linear chain of at least two carbon atoms with (b) an aldehyde having at least 16 carbon atoms, thereby providing the polyunsaturated fatty acid. In a further aspect, the polyunsaturated fatty acid has a chain length of at least 18 carbon atoms.

Examples of eye disorders include, but are not limited to, Stargardt-3 (STGD3) disease, macular dystrophy, age-related macular degeneration (AMD), and diabetic retinopathy.

In a further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the disorder.

In a further aspect, the eye disorder is selected from Stargardt-3 (STGD3) disease, macular dystrophy, age-related macular degeneration (AMD), and diabetic retinopathy. In a still further aspect, the eye disorder is STGD3 disease. In yet a further aspect, the eye disorder is macular dystrophy. In an even further aspect, the eye disorder is AMD. In a still further aspect, the eye disorder is diabetic retinopathy.

In a further aspect, the composition comprises an effective amount of the polyunsaturated fatty acid. In a still further aspect, the effective amount is a therapeutically effective amount. In yet a further aspect, the effective amount is a prophylactically effective amount.

In a further aspect, the method further comprises the step of administering a therapeutically effective amount of at least one agent known for the treatment of an eye disorder. Examples of agents known for treating eye disorders include, but are not limited to, fatty acids (e.g., docosapentaenoic acid, docosahexaenoic acid, eicosapentaenoic acid), anti-angiogenic agents, vitamin C, vitamin E, beta-carotene, zinc, and copper.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

### E. Methods for Supplementing a Female Subject's Diet

In one aspect, disclosed are methods for supplementing a female subject's diet, the method comprising administering to the female subject an effective amount of a disclosed composition, wherein the female subject is pregnant, desiring to become pregnant, or lactating. Thus, in various aspects, disclosed are methods for supplementing a female subject's diet, the method comprising administering to the female subject an effective amount of a composition comprising: (a) a liposome; (b) vitamin E; and (c) a polyunsaturated fatty acid having a chain length of at least 24 carbon atoms, or a pharmaceutically acceptable salt thereof, wherein the female subject is pregnant, desiring to become pregnant, or lactating.

In one aspect, disclosed are methods for supplementing a female subject's diet, the method comprising administering to the female subject an effective amount of a polyunsaturated fatty acid produced by a disclosed method, wherein the female subject is pregnant, desiring to become pregnant, or lactating. Thus, in various aspects, disclosed are methods for supplementing a female subject's diet, the method comprising administering to the female subject an effective amount of a polyunsaturated fatty acid produced by coupling, in the absence of heavy metals, (a) an activated alkyl halide having a protected alcohol and a linear chain of at least two carbon atoms with (b) an aldehyde having at least 16 carbon atoms, thereby providing the polyunsaturated fatty acid. In a further aspect, the polyunsaturated fatty acid has a chain length of at least 18 carbon atoms.

In various aspects, the female subject is pregnant. In a further aspect, the female subject is lactating.

In various aspects, the female subject is desiring to become pregnant. As used herein, the phrase “desiring to become pregnant,” indicates that the female subject is of child-bearing age (i.e., from puberty to menopause, typically from about twelve years of age to about fifty-one years of age), and also displays outward manifestations of an intent to become pregnant prior to and/or during the time period of administration. For example, the female subject may consume an agent recommended for use during pregnancy and/or lactation (e.g., fish oil, folic acid, vitamin D, vitamin C, calcium, thiamine, riboflavin, niacin, vitamin B12, fenugreek, fennel, palm dates, Coleus amboinicus) prior to and/or during administration of the disclosed composition. Alternatively, the female subject may be undergoing in vitro fertilization techniques. Additional outward manifestations of an intent to become pregnant are known by those of skill in the art.

In a further aspect, the method further comprises the step of identifying a female subject who is pregnant, desiring to become pregnant, or lactating.

In a further aspect, the composition comprises an effective amount of the polyunsaturated fatty acid. In a still further aspect, the effective amount is a neutraceutically effective amount.

In a further aspect, the method further comprises the step of administering a neutraceutically effective amount of at least one agent known for supplementing a female subject's diet, wherein the female subject is pregnant, desiring to become pregnant, or lactating. Examples of agents known for supplementing a female subject's diet include, but are not limited to, fish oil, folic acid, vitamin D, vitamin C, calcium, thiamine, riboflavin, niacin, vitamin B12, fenugreek, fennel, palm dates, and Coleus amboinicus.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

### F. Additional Methods of Using Fatty Acids and Compositions Containing Fatty Acids

The fatty acids and compositions of the invention are useful in treating or controlling eye disorders such as, for example, Stargardt-3 (STGD3) disease, macular dystrophy, age-related macular degeneration (AMD), and diabetic retinopathy. The fatty acids and compositions of the invention are also useful in supplementing a female subject's diet, wherein the female subject is pregnant, desiring to become pregnant, or lactating.

To treat or control the disorder or condition, the compounds and compositions comprising the fatty acids are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the fatty acids or compositions, the subject can be diagnosed with a need for treatment of an eye disorder or as being in need of diet supplementation, such as where the female subject is pregnant, desiring to become pregnant, or lactating.

The fatty acids or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of an eye disorder. A preparation can also be adminstered neutraceutically; that is, administered for diet supplementation or other physiological benefits.

The effective amount or dosage of the fatty acid can vary within wide limits. Such a dosage is adjusted to the individual requirements in each particular case including the specific fatty acid(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the fatty acid or composition to make up the daily dose. The dosage can be adjusted by the individual physician in the event of any contraindications. Dosage can vary, and can be administered in one or more dose administrations daily, for one or several days.

1. Use of Fatty Acids

In one aspect, the invention relates to the use of a disclosed fatty acid (e.g., a polyunsaturated fatty acid having a chain length of at least 24 carbon atoms) or a product of a disclosed method (e.g., a fatty acid having a chain length of at least 18 carbon atoms). In a further aspect, a use relates to the manufacture of a medicament for the treatment of an eye disorder. In a further aspect, a use relates to the manufacture of a medicament for supplementing a female subject's diet, wherein the female subject is pregnant, desiring to become pregnant, or lactating.

Also provided are the uses of the disclosed fatty acids and products. In one aspect, the invention relates to use of at least one disclosed fatty acid; or a pharmaceutically acceptable salt, hydrate, solvate, or polymorph thereof. In a further aspect, the fatty acid used is a product of a disclosed method of making.

In a further aspect, the use relates to a process for preparing a composition comprising an effective amount of a disclosed compound or a product of a disclosed method of making, or a pharmaceutically acceptable salt, solvate, or polymorph thereof, for use as a medicament.

In a further aspect, the use relates to a process for preparing a composition comprising an effective amount of a disclosed fatty acid or a product of a disclosed method of making, or a pharmaceutically acceptable salt, solvate, or polymorph thereof, wherein a pharmaceutically acceptable carrier is intimately mixed with a therapeutically effective amount of the fatty acid or the product of a disclosed method of making, or wherein a neutraceutically acceptable carrier is intimately mixed with a neutraceutically effective amount of the fatty acid or the product of a disclosed method of making.

In various aspects, the use relates to treatment of an eye disorder in a subject. In one aspect, the use is characterized in that the subject is a human. In one aspect, the use is characterized in that the eye disorder is Stargardt-3 (STGD3) disease, macular dystrophy, age-related macular degeneration (AMD), or diabetic retinopathy.

In various aspects, the use relates to supplementation of a female subject's diet, wherein the female subject is pregnant, desiring to become pregnant, or lactating.

In a further aspect, the use relates to the manufacture of a medicament for the treatment of an eye disorder.

In a further aspect, the use relates to the manufacture of a medicament for supplementation of a female subject's diet, wherein the female subject is pregnant, desiring to become pregnant, or lactating.

It is understood that the disclosed uses can be employed in connection with the disclosed fatty acids, products of disclosed methods of making, methods, compositions, and kits. In a further aspect, the invention relates to the use of a disclosed fatty acid or a disclosed product in the manufacture of a medicament for the treatment of an eye disorder of in a mammal. In a further aspect, the eye disorder is Stargardt-3 (STGD3) disease, macular dystrophy, age-related macular degeneration (AMD), or diabetic retinopathy. In a further aspect, the invention relates to the use of a disclosed fatty acid or a disclosed product in the manufacture of a medicament for supplementation of a female subject's diet, wherein the female subject is pregnant, desiring to become pregnant, or lactating.

2. Manufacture of a Medicament

In one aspect, the invention relates to a method for the manufacture of a medicament for treating an eye disorder in a subject having the disorder, the method comprising combining a therapeutically effective amount of a disclosed fatty acid or product of a disclosed method with a pharmaceutically acceptable carrier or diluent.

In one aspect, the invention relates to a method for the manufacture of a medicament for supplementing the diet of a female subject who is pregnant, desiring to become pregnant, or lactating, the method comprising combining a neutraceutically effective amount of a disclosed fatty acid or product of a disclosed method with a neutraceutically acceptable carrier or diluent.

As regards these applications, the present method includes the administration to an animal, particularly a mammal, and more particularly a human, of a therapeutically effective amount of the fatty acid effective in the treatment of an eye disorder, or of a neutraceutically effective amount of the fatty acid effective in supplementing the diet of a female subject who is pregnant, desiring to become pregnant, or lactating. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The total amount of the fatty acid of the present disclosure administered in a typical treatment is preferably between about 0.05 mg/kg and about 100 mg/kg of body weight for mice, and more preferably between 0.05 mg/kg and about 50 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing, and frequency of administration as well as the existence, nature and extent of any adverse side effects that might accompany the administration of the fatty acid and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed fatty acid or a product of a disclosed method of making, or a pharmaceutically acceptable salt, solvate, or polymorph thereof, with a pharmaceutically acceptable carrier or diluent, or with a neutraceutically acceptable carrier or diluent.

3. Kits

In one aspect, the invention relates to kits comprising an effective amount of a disclosed composition or a polyunsaturated fatty acid produced by a disclosed method, and one or more of: (a) an agent known for treating of an eye disorder; (b) an agent know for supplementing a female subject's diet, wherein the female subject is pregnant, desiring to become pregnant, or lactating; (c) instructions for administering the composition in connection with treating an eye disorder or supplementing a female subject's diet, wherein the female subject is pregnant, desiring to become pregnant, or lactating; and (d) instructions for treating an eye disorder or supplementing a female subject's diet wherein the female subject is pregnant, desiring to become pregnant, or lactating.

Thus, in one aspect, disclosed are kits comprising a composition comprising: (a) a liposome; (b) vitamin E; and (c) a polyunsaturated fatty acid having a chain length of at least 24 carbon atoms, or a pharmaceutically acceptable salt thereof, and one or more of: (d) an agent known for treating of an eye disorder; (e) an agent know for supplementing a female subject's diet, wherein the female subject is pregnant, desiring to become pregnant, or lactating; (f) instructions for administering the composition in connection with treating an eye disorder or supplementing a female subject's diet, wherein the female subject is pregnant, desiring to become pregnant, or lactating; and (g) instructions for treating an eye disorder or supplementing a female subject's diet wherein the female subject is pregnant, desiring to become pregnant, or lactating.

In one aspect, disclosed are kits comprising a polyunsaturated fatty acid produced by coupling, in the absence of heavy metals, (a) an activated alkyl halide having a protected alcohol and a linear chain of at least two carbon atoms with (b) an aldehyde having at least 16 carbon atoms, thereby providing the polyunsaturated fatty acid, and one or more of: (a) an agent known for treating of an eye disorder; (b) an agent know for supplementing a female subject's diet, wherein the female subject is pregnant, desiring to become pregnant, or lactating; (c) instructions for administering the composition in connection with treating an eye disorder or supplementing a female subject's diet, wherein the female subject is pregnant, desiring to become pregnant, or lactating; and (d) instructions for treating an eye disorder or supplementing a female subject's diet wherein the female subject is pregnant, desiring to become pregnant, or lactating. In a further aspect, the polyunsaturated fatty acid has a chain length of at least 18 carbon atoms.

In a further aspect, the composition comprises an effective amount of the polyunsaturated fatty acid. In a still further aspect, the effective amount is a therapeutically effective amount. In yet a further aspect, the effective amount is a prophylactically effective amount. In an even further aspect, the effective amount is a neutraceutically effective amount.

In a further aspect, the eye disorder is selected from Stargardt-3 (STGD3) disease, macular dystrophy, age-related macular degeneration (AMD), and diabetic retinopathy.

In a further aspect, the agent known for treating an eye disorder is selected from a fatty acid (e.g., docosapentaenoic acid, docosahexaenoic acid, eicosapentaenoic acid), an anti-angiogenic agent, vitamin C, vitamin E, beta-carotene, zinc, and copper.

In a further aspect, the composition and the agent known for treating an eye disorder are co-formulated. In a still further aspect, the composition and the agent known for treating an eye disorder are co-packaged.

In a further aspect, the polyunsaturated fatty acid and the agent known for treating an eye disorder are co-formulated. In a still further aspect, the polyunsaturated fatty acid and the agent known for treating an eye disorder are co-packaged.

In a further aspect, the agent known for supplementing a female subject's diet is selected from fish oil, folic acid, vitamin D, vitamin C, calcium, thiamine, riboflavin, niacin, vitamin B12, fenugreek, fennel, palm dates, and Coleus amboinicus.

In a further aspect, the composition and the agent known for supplementing a female subject's diet are co-formulated. In a still further aspect, the composition and the agent known for supplementing a female subject's diet are co-packaged.

In a further aspect, the polyunsaturated fatty acid and the agent known for supplementing a female subject's diet are co-formulated. In a still further aspect, the polyunsaturated fatty acid and the agent known for supplementing a female subject's diet are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

1. Materials and Methods

### a. Chemicals

All chemical reagents, such as methanol, hydrochloric acid, isopropanol, n-hexane, n-nonane, and diethyl ether, were of gas chromatography mass spectrometry (GC-MS) grade and purchased from Fisher Scientific (Pittsburgh, Pa., USA). All standards including the internal standards such as tridecanoic acid (13:0), hentriacontanoic acid (34:0), and all fatty acids methyl esters (FAMEs) such as methyl eicosanoate, methyl linolenate, methyl melissate, and Supelco-37 (a commercial mixture of FAMEs), alpha-tocopherol (VE) were purchased from Sigma-Aldrich (St. Louis, Mo., USA) and Matreya (Pleasant Gap, Pa., USA). Silica gel, glass-encased, solid-phase extraction cartridges (500 mg/6 ml) were purchased from Sorbent Technology (Atlanta, Ga., USA). The internal standards, tridecanoic acid and hentriacontanoic acid, were dissolved in nonane at concentrations of 1.0 mg/ml and 0.023 mg/ml, respectively. 1,2-distearoyl-sn-glycero-3-phosphocholine (DSPC) and 1,2-distearoyl-d₇₀-sn-3-glycero-phosphocholine (DSPCd₇₀)) were purchased from Avanti Polar Lipids (Alabaster, Ala.) and used without further purification. NaCl, NaOH, and HCl were purchased from Macron Chemicals (Center Valley, Pa.). Sodium phosphate anhydrous (dibasic), sodium phosphate (monobasic), and hydrogen peroxide were purchased from Fisher Scientific (Pittsburgh, Pa.). Chloroform and deuterium oxide were purchased from Sigma Aldrich Millipore (St. Louis, Mo.). PBS buffer consisting of 100 mM NaCl, 10 mM NaH₂PO₄, and 40 mM Na₂HPO₄ prepared in Nanopure water with a minimum resistivity of 18.2 MΩ·cm (Barnestead Thermolyne, Dubuque, Iowa) was used in all experiments. The pH was adjusted using either 2 M NaOH or 2 M HCl. Trapezoidal UV-IR grade fused silica trapezoidal prisms used as the membrane support were purchased from Almaz Optics (Marlton, N.J.) and cleaned thoroughly before use. Prisms were first placed in an UV-ozone cleaner (Jetlight, Co., Irvine, Calif.) for 30 minutes, followed by submersion in a solution of 30% (v/v) H₂O₂ and 70% (v/v) H₂SO₄ for a minimum of 30 minutes. (Caution: this is a highly corrosive solution that reacts violently with organic solvents. Take extreme caution and care when handling the solution.) The prisms were rinsed thoroughly with Nanopure water prior to drying in an oven at 120° C. for at least 15 minutes.

### b. Synthesis of (3-methyloxetan-3-yl)methyl 10-bromodecanoate (S1)

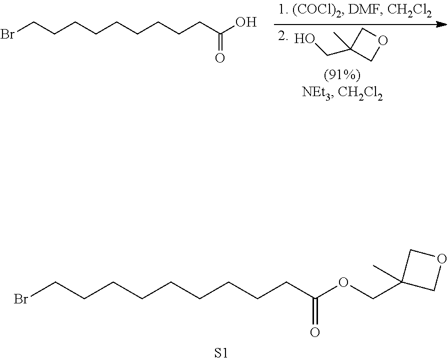

To a solution of 10-bromo-1-decanoic acid (2.60 g, 10.3 mmol) in CH₂Cl₂ (100 mL) at 0° C. was added 1 drop of DMF followed by oxalyl chloride (2.0 mL, 23.32 mmol). The resulting reaction mixture was allowed to warm to RT and stir for 2 h. The solvent was removed and the resulting residue containing 10-bromo-1-decanoyl chloride was used in the next step without additional purification.

To a solution of 10-bromo-1-decanoyl chloride from above in CH₂Cl₂ (75 mL) at 0° C. was added a solution of 3-methyl-3-oxetanemethanol (1.0 g, 9.8 mmol) and triethylamine (3.0 mL, 22 mmol) in CH₂Cl₂ (25 mL). This reaction mixture was allowed to warm to RT and stirred at that temperature for 3 hr after which the reaction was quenched with water (100 mL). The phases were separated and the aqueous layer was extracted with CH₂Cl₂ (3×50 mL). The combined organic extracts were washed with sat. NaHCO₃ (aq., 100 mL) and brine (100 mL), dried (Na₂SO₄), and concentrated. Flash chromatography (19:1 hexanes/ethyl acetate) gave 3.0 g (91%) of S1 as colorless oil. ¹H NMR (500 MHz, CDCl₃) δ 4.50 (d, J=5.8 Hz, 2H), 4.36 (d, J=5.8 Hz, 2H), 4.14 (s, 2H), 3.39 (t, J=6.8 Hz, 2H), 2.34 (t, J=7.3 Hz, 2H), 1.83 (qn, J=6.8 Hz, 2H), 1.63-1.61 (m, 2H), 1.40-1.39 (m, 2H), 1.32-1.29 (m, 11H); ¹³C NMR (125 MHz, CDCl₃) δ 174.0, 79.7, 68.6, 39.2, 34.3, 34.1, 32.9, 29.4, 29.3, 29.2, 28.8, 28.2, 25.1, 21.3; EI-LRMS calc'd for C₁₅H₂₇BrO₃ [M+H]⁺ (m/z) 335.1 found 335.1.

### C. Synthesis of 1-(9-bromononyl)-4-methyl-2,6,7-trioxabicyclo[2.2.2]octane (3)

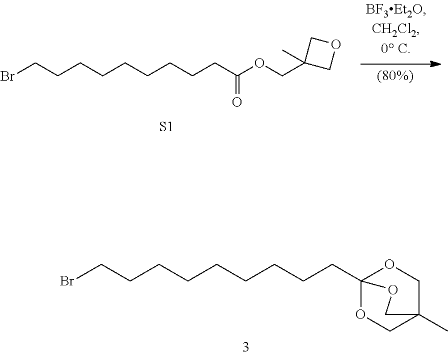 [SMILES: CC1(COC(=O)CCCCCCCCCBr)COC1 ; "S1" ; CC12COC(CCCCCCCCCBr)(OC1)OC2 ; "3"]

To a solution of S1 (3.0 g, 9.0 mmol) in CH₂Cl₂ (100 mL) at 0° C. was slowly added BF₃ OEt₂ (0.30 mL, 2.4 mmol). The resulting reaction mixture was warmed to RT and stirred at that temperature overnight after which it was cooled to 0° C. and the reaction was quenched by the addition of triethyl amine (1 mL). The resulting mixture was warmed to RT and to this was added water (50 mL). The phases were separated and the aqueous layer was extracted with CH₂Cl₂ (3×50 mL). The combined organic extracts were washed with sat. NaHCO₃ (aq., 50 mL) and brine (50 mL), dried (Na₂SO₄), and concentrated. Flash chromatography (19:1 ethyl acetate:hexane) gave 2.4 g (80%) of 3 as a thick colorless oil. ¹H NMR (500 MHz, CDCl₃) δ 3.88 (s, 6H), 3.38 (t, J=6.8 Hz, 2H), 1.83 (qn, J=6.8 Hz, 2H), 1.66-1.62 (m, 2H), 1.43-1.38 (m, 4H), 1.27-1.26 (m, 8H), 0.79 (s, 3H); ¹³C NMR (125 MHz, CDCl₃) δ 109.2, 72.7, 36.9, 34.2, 33.0, 30.4, 29.6, 29.5, 29.4, 28.9, 28.3, 23.3, 14.7; IR (neat) 2925, 2853, 1733, 1458, 1397, 1352, 1261, 1055, 990, 886 cm⁻¹; EI-LRMS calcd for C₁₅H₂₇BrO₃ [M+H]⁺ (m/z) 335.1. found 335.1.

### d. Synthesis of (4Z,7Z,10Z,13Z,16Z,19Z)-docosa-4,7,10,13,16,19-hexanal (5)

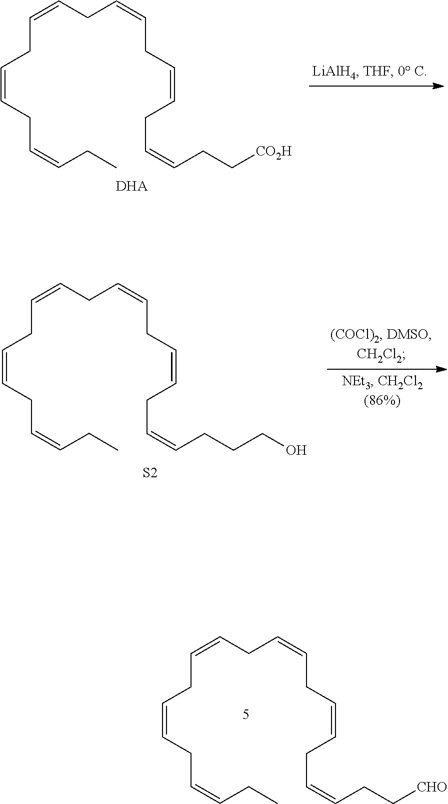

To a 0.2 M solution of DHA in dry THF at 0° C. was added LiAlH₄ (4.1 equivalents relative to DHA). The resulting solution was stirred at 0° C. until the reaction was judged complete by TLC (ca. 2 h). The reaction was quenched with saturated aqueous (sat'd. aq.) Na₂SO₄ (0.004 equivalents relative to DHA). The resulting mixture was warmed to rt and stirred for an additional 1 h. The solution was filtered through celite and the solvent was removed under reduced pressure to give DHA alcohol in 97% yield that was used in the subsequent oxidation reaction without additional purification.

To a 0.5 M solution of DMSO (8.1 equivalents relative to DHA alcohol) in CH₂Cl₂ at −78° C. was slowly added oxalyl chloride (8.7 equivalents relative to DHA alcohol). After the resulting mixture had stirred for 0.5 h, a 0.5 M solution of DHA alcohol in CH₂Cl₂ was then added slowly. The resulting mixture was stirred for an additional 2 h. To this was slowly added a 6.4 M solution of triethyl amine (17 equivalents relative to DHA alcohol) in CH₂Cl₂ over a 1 h time period. Once the addition was completed the solution was stirred at −78° C. for an additional 1 h and then warmed to rt. The reaction was quenched by adding water (equivalent volume to CH₂Cl₂). The layers were separated and the aqueous phase was extracted three times with CH₂Cl₂. The combined organic layers were washed with brine, dried (Na₂SO₄) and concentrated under reduced pressure. The product was purified by flash column chromatography using silica gel that had been neutralized using 2% Et₃N. The eluent for the purification consisted of a mixture of ethyl acetate and hexanes giving aldehyde 5 in 84% yield. ¹H NMR (500 MHz, CDCl₃) δ 3.88 (s, 6H), 3.38 (t, J=6.8 Hz, 2H), 1.83 (qn, J=6.8 Hz, 2H), 1.66-1.62 (m, 2H), 1.43-1.38 (m, 4H), 1.27-126 (m, 8H), 0.79 (s, 3H); ¹³C NMR (125 MHz, CDCl₃) δ 202.0, 132.2, 129.5, 128.7, 128.5, 128.4, 128.2, 128.1, 128.0, 127.8, 127.2, 43.8, 25.8, 25.7, 20.7, 20.2, 14.4; IR (neat) 3011, 2962, 2921, 2714, 1726, 1658, 1434, 1390, 1352, 1265, 1055, 917, 702 cm⁻¹; EI-LRMS calcd for C₁₅H₂₇BrO₃ [M+Na]⁺ (m/z) 335.5. found 335.3.

### e. Synthesis of 1-((13Z,16Z,19Z,22Z,25Z,28Z)-hentriaconta-13,16,19,22,25,28-hexaen-1-yl)-4-methyl-2,6,7-trioxabicyclo[2.2.2]octane (9)

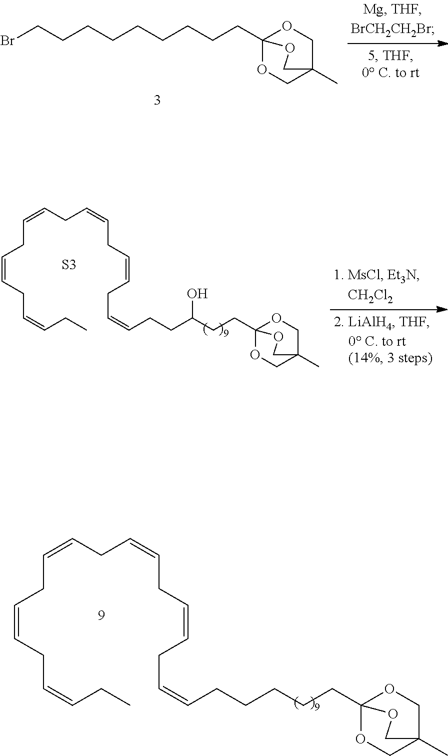

To a flask charged with magnesium powder (100. mg, 4.10 mmol) at RT was added 1 drop of dibromoethane followed by a solution of orthoester 3 (800. mg, 2.40 mmol) in THF (15 mL). The reaction mixture was stirred for 2 hr and then cooled to 0° C. To this was added a solution of DHA aldehyde 5 (80.0 mg, 0.250 mmol) in THF (5 mL). The resulting mixture was warmed to RT and stirred at that temperature for 2 hr after which the reaction was quenched with sat. NH₄C1 (aq., 20 mL). The phases were separated and the aqueous phase was extracted with EtOAc (3×20 mL). The extracts were combined, dried (Na₂SO₄), and concentrated. Flash chromatography (a gradient of 1:9 to 2:8 ethyl acetate:hexanes) gave 345 mg that consisted of an inseparable mixture of alcohol S3 and the dimer from orthoester 3 as a white solid.

To a solution of the mixture from above (345 mg) in CH₂Cl₂ (10 mL) at 0° C. was added triethylamine (0.3 mL, 2 mmol) and MsCl (50 μL, 0.65 mmol). The resulting reaction mixture was warmed to RT and stirred at that temperature for 2 hr after which the reaction was quenched with water (10 mL). The aqueous phase was extracted with CH₂Cl₂ (3×10 mL) and filtered through Na₂SO₄. Concentration and flash chromatography (1:4 ethyl acetate:hexanes) gave 300 mg of a mixture that consisted of mesylated S3 and the dimer from orthoester 3 as a light yellow oil.

To solution of the mixture from above (300 mg) in THF (15 mL) was added LiAlH₄ (150 mg, 3.95 mmol) at 0° C. The resulting reaction mixture was warmed to RT and stirred at that temperature for 8 hr after which the reaction was quenched with sat. Na₂SO₄ (aq., 2 mL). The resulting mixture was stirred for 1 hr and then filtered and concentrated. Flash chromatography (1:9 ethyl acetate:hexanes) gave 20 mg (14%, 3 steps) of 9 as a colorless oil. ¹H NMR (500 MHz, CDCl₃) δ 5.42-5.30 (m, 12H), 3.89 (s, 6H), 2.84-2.80 (m, 10H), 2.09-2.02 (m, 4H), 1.67-1.63 (m, 2H), 1.43-1.24 (m, 22H), 0.97 (t, J=7.3 Hz, 3H), 0.79 (s, 3H); ¹³C NMR (125 MHz, CDCl₃) δ 132.2, 130.7, 128.8, 128.7, 128.5, 128.4, 128.3, 128.0, 127.7, 127.2, 109.3, 72.7, 36.9, 30.4, 29.9, 29.8, 29.7, 29.5, 27.4, 25.8, 25.7, 23.3, 20.7, 14.7, 14.4.

### f. Synthesis of (14Z,17Z,20Z,23Z,26Z,29Z)-dotriaconta-14,17,20,23,26,29-hexaenoic Acid (1)

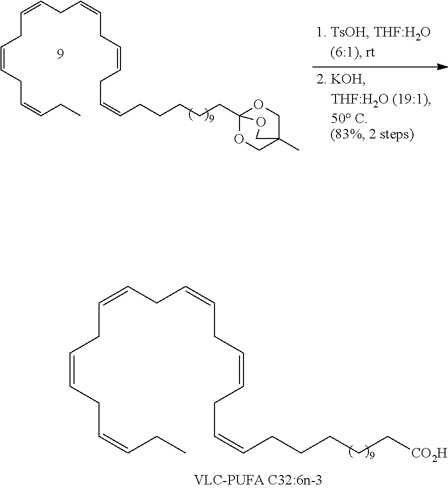

To a mixture of 9 (20. mg, 0.036 mmol), THF (5 mL), and H₂O (0.5 mL) at RT was added TsOH H₂O (0.6 mg, 0.003 mmol). The resulting reaction mixture was stirred overnight after which it was concentrated and used in the next step without additional purification.

A solution of the crude ester from above (30 mg) in THF (5 mL) and 15% NaOH (aq., 0.2 mL) was stirred for 1 hr at 50° C. The reaction was quenched with 1M HCl (aq., 5 mL) and the aqueous phase was extracted with EtOAc (3×5 mL). The extracts were combined, dried (Na₂SO₄), and concentrated. Flash chromatography (1:4 EtOAc:hexanes) gave 14 mg (83%) of VLC-PUFA C32: 6n-3.

### g. Synthesis of 2-((10-bromodecyl)oxy)tetrahydro-2H-pyran (2)

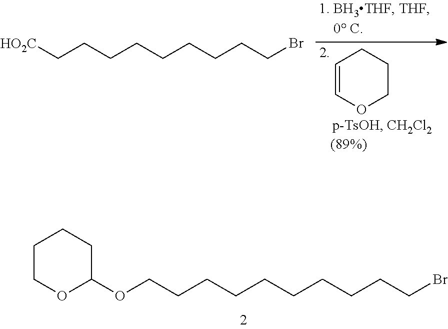 [SMILES: O=C(O)CCCCCCCCCBr ; C1=COCCC1 ; BrCCCCCCCCCCOC1CCCCO1]

To a solution of 10-bromodecanoic acid (5.0 g, 0.39 mmol) in THF (100 mL) at 0° C. was added BH₃ THF (30 mL of a 1.0 M in THF, 30 mmol) over 1 hr dropwise. The reaction mixture was then warmed to RT and stirred for 4 hr after which it was cooled back down to 0° C. The reaction was quenched by the addition of a 1:1 (v/v) mixture of THF:water (100 mL). The phases were separated and the aqueous phase was extracted with ether (3×25 mL). The organic extracts were combined, washed with brine (50 mL), dried (Na₂SO₄), and concentrated. The resulting residue that contained 10-bromo-1-decanol was used without further purification.

To a solution of 10-bromo-1-decanol from above and dihydropyran (3.80 mL, 32.9 mmol) in CH₂Cl₂ (100 mL) at 0° C. was added p-toluene sulfonic acid monohydrate (0.38 g, 2.0 mmol). The resulting reaction mixture was warmed to RT and stirred overnight after which the reaction was quenched with H₂O (50 mL). The phases were separated and the aqueous phase was extracted with CH₂Cl₂ (3×25 mL). The organic extracts were combined, dried (Na₂SO₄), and concentrated. Flash column chromatography (1:19 EtOAc:hexanes) gave 5.7 g of bromide 2 (89%) as a colorless oil.

R_{f}=0.57 (1:9 EtOAc:hex); ¹H NMR (800 MHz, CDCl₃) δ 4.57 (dd, J=4.3, 3.2 Hz, 1H), 3.87 (ddd, J=11.1, 8.0, 3.0 Hz, 2H), 3.72 (dt, J=9.6, 6.9 Hz, 1H), 3.52-3.47 (m, 1H), 3.40 (t, J=6.9 Hz, 2H), 3.37 (dt, J=9.6, 6.8 Hz, 1H), 1.88-1.79 (m, 2H), 1.71 (ddt, J=12.8, 9.2, 3.3 Hz, 1H), 1.63-1.54 (m, 2H), 1.54-1.49 (m, 2H), 1.44-1.38 (m, 3H), 1.34 (q, J=7.0 Hz, 1H), 1.32-1.24 (m, 10H); ¹³C NMR (126 MHz, CDCl₃) δ 99.0, 67.8, 62.4, 34.0, 33.0, 30.9, 29.9, 29.6, 29.5, 29.5, 28.8, 28.3, 26.3, 25.6, 19.8; IR (neat) 2924, 2853, 1735, 1465, 1440, 1352, 1322, 1260, 1200, 1183, 1163, 1135, 1120, 1078, 1032, 988, 905, 869, 815, 722, 646, 564 cm⁻¹; EI-LRMS calcd for C₁₅H₂₉O₂Br [M+Na]⁺ (m/z) 344.29. found 345.0.

### h. Synthesis of (14Z,17Z,20Z,23Z,26Z,29Z)-1-((tetrahydro-2H-pyran-2-yl)oxy)dotriaconta-14,17,20,23,26,29-hexaen-11-ol (6)

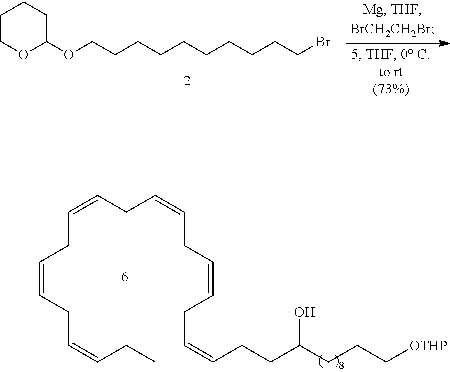

A 100 mL round bottom flask was charged with dry magnesium turnings (300. mg, 12.3 mmol). To this was added bromide 2 (2.00 g, 6.23 mmol) in THF (12 mL) and a catalytic amount of dibromoethane. The reaction was allowed to stir at RT for 2 h. The formation of the Grignard reagent was monitored by ¹H NMR for the disappearance of the CH₂ signal at 3.40 ppm. Once the signal had disappeared, the reaction mixture was cooled to 0° C. and to it was added DHA aldehyde 5 slowly. After the addition was complete, the mixture was warmed to RT and stirred for an additional 2 h. The reaction was quenched with sat. NH₄Cl (aq., 25 mL), the phases were separated, and the aqueous phase was extracted with ether (3×25 mL). The organic extracts were combined, dried (Na₂SO₄) and concentrated. Flash chromatography (1:9 EtOAc:hexanes) gave 550 mg of alcohol 6 (73%) as a colorless oil.

R_{f}=0.66 (1:4 EtOAc:hexanes); ¹H NMR (500 MHz, CDCl₃) δ 5.51-5.21 (m, 12H), 4.57 (t, J=4.2, 2.9 Hz, 1H), 3.86 (ddd, J=11.1, 7.6, 3.5 Hz, 1H), 3.72 (dt, J=9.6, 6.9 Hz, 1H), 3.60 (tt, J=8.7, 4.4 Hz, 1H), 3.54-3.44 (m, 1H), 3.37 (dt, J=9.6, 6.7 Hz, 1H), 2.90-2.75 (m, 10H), 2.27-2.12 (m, 2H), 2.11-2.03 (m, 2H), 1.89-1.76 (m, 1H), 1.75-1.66 (m, 1H), 1.63-1.47 (m, 5H), 1.47-1.18 (m, 20H), 0.97 (t, J=7.5 Hz, 3H); ¹³C NMR (126 MHz, CDCl₃) δ 132.2, 130.0, 128.7, 128.5, 128.4, 128.4, 128.3, 128.2, 128.2, 128.0, 127.1, 99.0, 71.7, 67.8, 62.5, 37.7, 37.3, 30.9, 29.9, 29.8, 29.8, 29.7, 29.7, 29.6, 26.4, 25.8, 25.8, 25.8, 25.7, 25.6, 23.7, 20.7, 19.8, 14.4; IR (neat) 3396, 3012, 2925, 2853, 1653, 1454, 1391, 1353, 1323, 1262, 1200, 1184, 1136, 1121, 1077, 1023, 987, 906, 868, 814, 719 cm⁻¹; HRMS calcd for C₃₇H₆₂O₃ [M+Na]⁺ (m/z) 577.4597. found 577.6.

### i. Synthesis of 2-(((14Z,17Z,20Z,23Z,26Z,29Z)-dotriaconta-14,17,20,23,26,29-hexaen-1-yl)oxy)tetrahydro-2H-pyran (8)

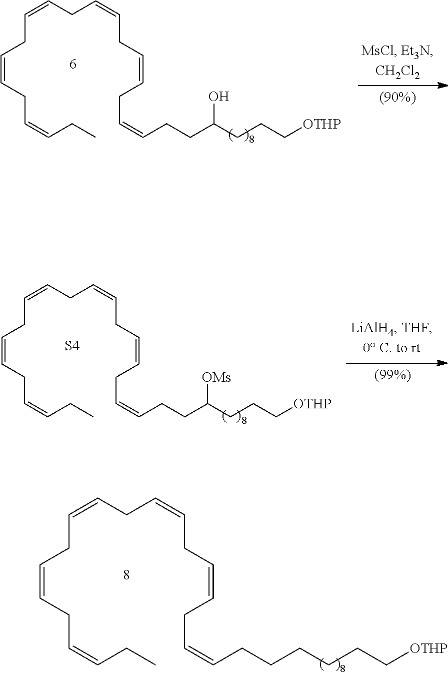

(14Z,17Z,20Z,23Z,26Z,29Z)-1-((tetrahydro-2H-pyran-2-yl)oxy)dotriaconta-14,17,20,23,26,29-hexaen-11-yl methanesulfonate (S4). To a solution of alcohol 6 (1.17 g, 2.11 mmol) and triethyl amine (2.8 mL, 20. mmol) in CH₂Cl₂ (27 mL) at 0° C. was added methanesulfonylchloride (0.50 mL, 6.3 mmol) slowly. The reaction mixture was warmed to RT and stirred at that temperature for 2 hr before the reaction was quenched with water (20 mL). The phases were separated and the aqueous phase was extracted with CH₂Cl₂ (3×20 mL). The organic extracts were combined, washed with brine (50 mL), dried (Na₂SO₄), and concentrated. Flash chromatography (1:9 EtOAc:hexanes) gave 1.20 g of mesylate S4 (90%) as a colorless oil.

R_{f}=0.54 1:4 EtOAc:hexanes; ¹H NMR (800 MHz, CDCl₃) δ 5.44-5.28 (m, 12H), 4.72 (d, J=6.1 Hz, 1H), 4.57 (dd, J=4.2, 3.2 Hz, 1H), 3.86 (ddd, J=11.2, 8.0, 3.1 Hz, 1H), 3.72 (dt, J=9.6, 6.9 Hz, 1H), 3.53-3.46 (m, 1H), 3.37 (dt, J=9.5, 6.7 Hz, 1H), 2.99 (s, 3H), 2.89-2.76 (m, 10H), 2.23-2.12 (m, 2H), 2.07 (p, J=7.5 Hz, 2H), 1.86-1.80 (m, 1H), 1.79-1.64 (m, 4H), 1.63-1.54 (m, 3H), 1.54-1.49 (m, 2H), 1.46-1.22 (m, 14H), 0.97 (t, J=7.5 Hz, 3H), 2.23-2.11 (m, 2H); ¹³C NMR (101 MHz, CdCl₃) δ 132.2, 129.2, 128.7, 128.6, 128.4, 128.4, 128.3, 128.2, 128.2, 128.2, 128.0, 127.1, 99.0, 83.7, 67.8, 62.5, 38.8, 34.6, 34.5, 30.9, 29.9, 29.7, 29.6, 29.6, 29.5, 26.4, 25.8, 25.8, 25.7, 25.6, 25.1, 23.0, 20.7, 19.8, 14.4; IR (neat) 3439, 3012, 2923, 2852, 1722, 1455, 1349, 1171, 1118, 1075, 1022, 970, 901, 721 cm⁻¹; HRMS calcd for C₃₈H₆₄O₅S [M+Na]⁺ (m/z) 655.4372. found 655.4367.

2-(((14Z,17Z,20Z,23Z,26Z,29Z)-dotriaconta-14,17,20,23,26,29-hexaen-1-yl)oxy)tetrahydro-2H-pyran (8). To a solution of mesylate S4 (0.40 g, 0.63 mmol) in THF (20 mL) at 0° C. was added LiAlH₄ (0.26 g, 6.8 mmol). The resulting mixture was warmed to RT and stirred at that temperature for 8 h. The reaction mixture was then cooled back to 0° C. and the reaction was quenched with sat. Na₂SO₄ (aq., 5 mL). The mixture was stirred at RT for 1 hr and the resulting slurry was filtered through celite and concentrated to give 0.34 g of 8 (99%) as a thick pale yellow oil which was used without additional purification.

¹H NMR (500 MHz, CDCl₃) δ 5.44-5.27 (m, 12H), 4.57 (dd, J=4.2, 3.0 Hz, 1H), 3.87 (ddd, J=11.1, 7.8, 3.2 Hz, 1H), 3.73 (dt, J=9.6, 6.9 Hz, 1H), 3.53-3.45 (m, 1H), 3.38 (dt, J=9.5, 6.7 Hz, 1H), 2.91-2.76 (m, 10H), 2.13-2.01 (m, 4H), 1.90-1.77 (m, 1H), 1.75-1.66 (m, 1H), 1.64-1.47 (m, 6H), 1.41-1.18 (m, 20H), 0.97 (t, J=7.5 Hz, 3H); ¹³C NMR (126 MHz, CDCl₃) δ 132.1, 130.6, 128.7, 128.7, 128.4, 128.3, 128.2, 128.0, 128.0, 127.6, 127.1, 98.9, 67.8, 62.4, 30.9, 29.9, 29.8, 29.8, 29.8, 29.7, 29.6, 29.5, 27.4, 26.4, 25.8, 25.8, 25.7, 20.7, 19.8, 14.4; EI-LRMS calcd for C₃₇H₆₂O₂Na [M+Na]⁺ (m/z) 561.4648. found 561.4645.

### j. Synthesis of (14Z,17Z,20Z,23Z,26Z,29Z)-dotriaconta-14,17,20,23,26,29-hexaenoic Acid (1)

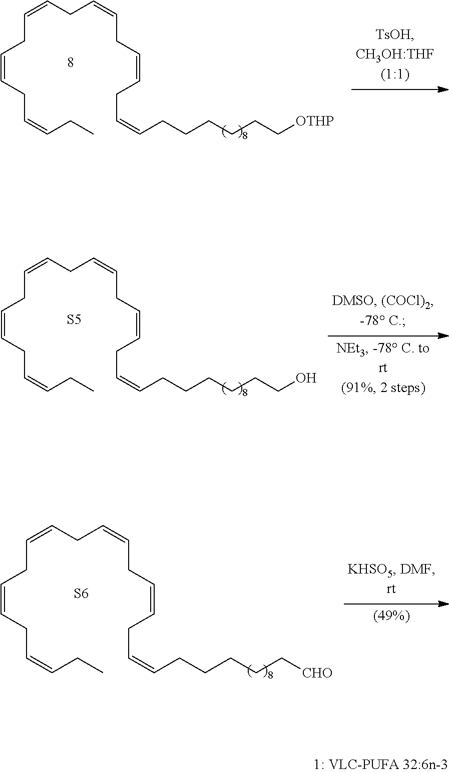

(14Z,17Z,20Z,23Z,26Z,29Z)-DOTRIACONTA-14,17,20,23,26,29-HEXAEN-1-OL (S3). To a solution of 8 in CH₃OH (6 mL) and THF (6 mL) at RT was added p-toluene sulfonic acid monohydrate (12 mg, 0.060 mmol). The reaction mixture was stirred overnight and then concentrated. Flash chromatography (1:9 EtOAc:hexanes) gave 0.21 g of S5 (75%) as a colorless oil.

R_{f}=0.3 1:9 (EtOAc:hexanes); ¹H NMR (500 MHz, CDCl₃) δ 5.47-5.26 (m, 12H), 3.64 (t, J=6.6 Hz, 2H), 2.92-2.75 (m, 10H), 2.07 (dq, J=14.4, 7.2 Hz, 4H), 1.56 (p, J=6.7 Hz, 3H), 1.41-1.19 (m, 20H), 0.98 (t, J=7.5 Hz, 3H); ¹³C NMR (126 MHz, CDCl₃) δ 132.2, 130.6, 128.8, 128.7, 128.4, 128.4, 128.3, 128.0, 128.0, 127.7, 127.2, 63.2, 33.0, 29.8, 29.8, 29.8, 29.8, 29.8, 29.7, 29.6, 29.5, 27.4, 25.9, 25.8, 25.8, 25.7, 20.7, 14.4; IR (neat) 3327, 3013, 2923, 2852, 2362, 2338, 1653, 1457, 1393, 1266, 1056, 925, 719, 668; HRMS calculated for C₃₂H₅₅O [M+H]⁺ (m/z) 455.4253. found 455.4250.

(14Z,17Z,20Z,23Z,26Z,29Z)-dotriaconta-14,17,20,23,26,29-hexanal (S6). To a solution of DMSO (1.40 mL, 19.71 mmol) in CH₂Cl₂ (20 mL) at −78° C. was added oxalyl chloride (0.82 mL, 9.69 mmol) dropwise over a 1 hr time period. The resulting solution was stirred for an additional 2 hr after which S5 (431 mg, 0.971 mmol) was added dropwise. The reaction mixture was further stirred for another 2 hr and then a solution of triethyl amine (2.8 mL, 20. mmol) in CH₂Cl₂ (3 mL) was added dropwise. The reaction mixture was stirred for 1 hr then it was warmed to RT. The reaction was quenched with H₂O (25 mL), the phases were separated, and the aqueous phase was extracted with CH₂Cl₂ (3×25 mL). The organic extracts were combined, dried (Na₂SO₄), and concentrated. Flash chromatography (1:19 ethyl acetate:hexanes) gave 0.50 g of S4 (91%) as a pale yellow oil.

R_{f}=0.4 (1:9 EtOAc:hexanes); ¹H NMR (500 MHz, CDCl₃) δ 9.74 (t, J=1.7 Hz, 1H), 5.58-5.15 (m, 12H), 2.94-2.69 (m, 10H), 2.44-2.36 (m, 2H), 2.11-1.99 (m, 4H), 1.61 (p, J=7.2 Hz, 2H), 1.41-1.17 (m, 18H), 0.96 (t, J=7.5 Hz, 3H); ¹³C NMR (126 MHz, CDCl₃) δ 202.7, 132.0, 130.5, 128.6, 128.6, 128.3, 128.3, 128.2, 127.9, 127.9, 127.6, 127.1, 44.0, 29.8, 29.8, 29.7, 29.7, 29.6, 29.6, 29.5, 29.4, 29.4, 29.3, 27.3, 25.9, 25.7, 25.7, 25.6, 22.2, 20.6, 14.4, 14.2; IR (neat) 3012, 2960, 2921, 2714, 1722, 1658, 1434, 1390, 1352, 1265, 1065, 917, 702 cm⁻¹.

(14Z,17Z,20Z,23Z,26Z,29Z)-dotriaconta-14,17,20,23,26,29-hexaenoic acid (1). To a solution of aldehyde S6 (0.500 g, 1.11 mmol) in DMF (11 mL) at RT was added KHSO₅ (0.76 g, 1.2 mmol) in one portion. After stirring for 3 hours, the mixture was filtered through a short plug of silica gel and the eluent was concentrated. Flash chromatography (1:4 EtOAc:hexanes) gave 0.25 g of carboxylic acid 1 (49%) as a thick pale yellow oil. ¹H NMR (500 MHz, CDCl₃) δ 10.70 (s, 1H), 5.47-5.25 (m, 12H), 2.90-2.76 (m, 10H), 2.35 (t, J=7.5 Hz, 2H), 2.12-2.00 (m, 4H), 1.63 (p, J=7.4 Hz, 2H), 1.39-1.20 (m, 18H), 0.97 (t, J=7.5 Hz, 3H); ¹³C NMR (101 MHz, CDCl₃) δ 179.6, 132.2, 130.6, 128.7, 128.4, 128.3, 128.0, 127.7, 127.2, 34.1, 29.8, 29.8, 29.6, 29.5, 29.4, 29.2, 27.4, 25.8, 25.7, 24.8, 20.7, 14.4; IR (neat) 3012, 2923, 2853, 2361, 2337, 1708, 1650, 1457, 1267, 1069, 926, 719, 668 cm⁻¹; HRMS calculated for C₃₂H₅₂O₂Na [M+Na]⁺ (m/z) 491.3865. found 491.3857.

### k. Π-A Isotherms

Pressure-area (π-A) isotherms were collected using a Langmuir trough (KSV, Helsinki, Finland). PBS was used as the subphase for all isotherms. Stock solutions of pure VLC-PUFA, pure DSPC, and DSPC containing 0.1 mol %, 1 mol %, or 10 mol % VLC-PUFA were spread at the air/water interface and allowed to equilibrate for 15 minutes prior to compression to allow for solvent evaporation. The monolayers were compressed at a rate of 2 mm/min at 22±1° C. and repeated in triplicate. The averaged isotherms were smoothed using a 100-point rolling average. The MMA of mixture of VLC-PUFA in DSPC layers was calculated by the formula: MMA_{DSPC+VLC-PUFA}=MMA_{DSPC}X_{DSPC}+MMA_{VLC-PUFA}X_{VLC-PUF}A, where the X is mol fraction of DSPC and VLC-PUFA in membrane.

### l. Bilayer Preparation

Planar supported lipid bilayers (PSLBs) used in this study were prepared using the Langmuir-Blodgett/Langmuir-Schaefer (LB/LS) method, the details of which are presented elsewhere (Liu and Conboy (2009) Vib. Spectrosc. 50: 106-115). Briefly, the cleaned SiO₂ prisms were submerged in PBS. Afterwards, 0.1 mol % VLC-PUFA in DSPCd₇₀ was spread over the air/water interface and allowed to thermally equilibrate for 15 minutes. Afterwards, the monolayer was compressed to 30 mN/m at a rate of 4 mm/min, and the surface pressure was allowed to stabilize for another 5 minutes. The prism was then withdrawn at a rate of 3 mm/min to deposit the LB layer. The subphase was removed, and the trough was cleaned using methanol, isopropanol, and water. Following this, fresh PBS was introduced into the trough, and 0.1 mol % VLC-PUFA in DSPC was spread at the air/water interface. The monolayer was then compressed to 30 mN/m at a rate of 4 mm/min. The prism was rotated horizontally so that the LB layer was parallel to the air/water interface. The prism was submerged through the monolayer, depositing the LS layer resulting in the formation of a fully assembled bilayer. From this point forward, the bilayer was maintained in an aqueous environment. The prism was transferred to a custom Teflon flow-cell that was equipped with ports to allow for solution exchange and a K-type thermocouple to monitor the temperature. A surface pressure of 30 mN/m was chosen in order to reflect the pressure of the plasma membrane in cells and solution phase vesicles.

### m. DSPC Flip-Flop Kinetics

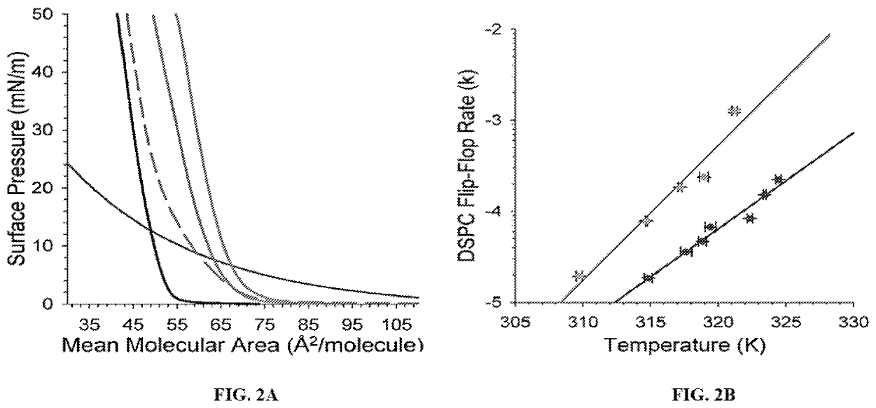

Following bilayer assembly, the flow-cell was flushed with PBS in D₂O to eliminate any spectral interference from the 0-H bands from water. The spectrometer setup used in these studies is described in detail elsewhere (Allhusen, et al. (2016) J. Phys. Chem. B 120: 3157-3168). An initial spectrum was taken prior to kinetics measurements to ascertain bilayer formation. The spectrometer was then tuned to 2875 cm⁻¹, and the temperature of the flow cell was raised for the kinetics measurements. The flip-flop rates of DSPC were determined from the decay of methyl symmetric stretch mode (CH₃ vs) for an asymmetrically prepared VLC-PUFA:DSPC membrane in which on leaflet contained dueterated DSPCd₇₀ and DSPC. As DSPC lipid exchange between leaflets, the intensity of the CH₃ vs decreases over time due to flip-flop and the rate of exchange is determined by fitting the data to I_{CH}_{₃}(t)=Iₘᵢₙ+Iₘₐₓe^{-4kt}, the results of which are presented in FIG. 2B. The half-lives of flip-flop were calculated using

$${t}_{1 / 2} = \frac{ln ( 2 )}{2 k}$$

and also are listed in FIG. 2B.

### n. Animals

WT mice (C57BL/6J) mice have been used in this study for single dose gavage, repeated dose gavage and optomotry experiments. VLC-PUFA liposomes have been prepared using liposome kit (0.05%) with an addition of alpha-tocopherol (VE) (0.025%) to prevent oxidation. Three month old WT mice were gavage fed with VLC-PUFA liposomes. A dose of 6 mg/mice/day was used, half the dosage used for DHA by previous studies (Jiang, et al. (2009) The Journal of Nutritional Biochemistry 20: 735-741; Hacioglui, et al. (2007) Neurobiology of Learning and Memory 87: 159-165). The mice were gavage fed and sacrificed at time intervals (n=4/time point) of 2, 4, 8, and 24 hr to harvest the blood and other tissues. Eyes were dissected under the microscope to separate retina from RPE. One pair of retina/mouse have been used for lipid extraction as described in the procedure elsewhere. Blood was centrifuged for 5 min at 7500 rpm to separate serum from RBC. Serum, RBC, liver, retina and RPE have been used for lipid extractions and GC-MS analysis.

Repeated dose gavage. VLC-PUFA liposomes were gavaged to three month old WT mice for 15 days. The mice were divided into three groups (n=4/group), Group 1 was fed with liposomes prepared with PC and alpha-tocopherol, Group 2 was fed with liposomes prepared with PC, VE, and 1 mg VLC-PUFAs and Group 3 was fed with liposomes with PC, VE and 2 mg VLC-PUFAs. Mice were sacrificed at the end of the experiment and organs were harvested, extracted for lipids and analyzed by GC-MS using the procedure mentioned elsewhere.

Optomotry/Visual behavior test. Repeated dose gavage fed mice were tested for visual behavior to observe the effect of VLC-PUFA on vision. Since the optomotry tests could be manipulative, all the analysts were double blinded. 3 month-old mice were employed to test spatial visual acuity using the OptoMotry system (Cerebral Mechanics, Lethbridg, AB, Canada). Briefly, individual mice were placed on a platform centered in a quad-square formed by four inward facing computer screens, and their movements were monitored by an overhead video camera. Photopic measurements were conducted under illuminance of around 165 lux. Scotopic measurements were carried out in infrared light with the LCD displays masked with 5 layers of ND16 Lee299 filters. During the detection of spatial frequency threshold, the rotation speed and contrast were kept at 12°/s, and 100%, respectively, while the frequency was kept at 0.19 cycle/degree in the examination of contrast sensitivity. All experiments had concurrent control mice fed with placebo chow.

### O. Lipid Extraction and Purification of Lipids with Solid-Phase Extraction

Serum, RBCs, orbital adipose tissue, and retina punches, were extracted using the procedure previously adopted (Liu, et al. (2013) J. Chromatogr. A 1307: 191-200). The samples and internal standards (50 μg of tridecanoic acid and 1.15 μg of hentriacontanoic acid) were added in 2 ml stainless steel vials and then homogenized with 0.7 ml silica beads and 1 ml hexane-isopropanol (3:2 v:v) by a Mini Bead Beater-16 (BioSpec Products Inc., Bartlesville, Okla., USA) and a Sonic Dismembrator Model 100 (Fisher Scientific, Pittsburgh, Pa., USA). The homogenized samples were bath sonicated for 5 min in an ice water bath. After centrifugation at 10,000 rpm for 5 min, the extracted solution supernatant was transferred to a clean vial and then dried under a stream of nitrogen. The dried film was dissolved in 200 μl hexane, and 2 ml of 4% HCl in methanol was added. The tubes were flushed with argon and incubated at 80° C. for 4 hr to form FAMEs (Liu, et al. (2013) J. Chromatogr. A 1307: 191-200) and then allowed to cool. The FAME mixture was extracted three times with 1 ml distilled water and 2 ml hexane. The hexane layers were combined and dried under nitrogen gas.

Silica gel, glass-encased, solid-phase extraction cartridges were subsequently used to clean the FAME extracts. The cartridge was activated with 6 ml of hexane before loading samples. The crude FAME extract was dissolved in 200 μl of hexane and loaded onto the activated cartridge. The cartridge was washed with 6 ml hexane, and the eluate was discarded. FAMEs were eluted from the cartridge with 5 ml hexane:ether (4:1), and the eluate was evaporated under nitrogen gas. The dry film was dissolved in 200 μl of hexane and centrifuged for 3 min at 14,000 rpm to remove particles prior to GC-MS analysis. 1 μl of sample was injected into the GC-MS instrument for LC-PUFAs analysis. For VLC-PUFAs analysis, the sample was dried with nitrogen again and re-dissolved in 20 μl of n-nonane, and 5 μl samples were injected into the GC-MS instrument.

### p. GC-MS Instrumentation and Chromatographic Conditions

The Thermo Trace GC-DSQ II system (ThermoFisher Scientific, Waltham, Mass., USA) consists of an automatic sample injector (AS 3000), gas chromatograph (GC), single quadrupole mass detector, and an analytical workstation. The chromatographic separation was carried out with an Rxi-5MS coated 5% diphenyl/95% dimethyl polysiloxane capillary column (30 m×0.25 mm i.d, 0.25 μm film thickness) (Restek, Bellefonte, Pa., USA). Two methods (A and B) were used for detection and quantitation of LC-PUFAs and VLC-PUFAs, respectively as previously published (Liu, et al. (2013) J. Chromatogr. A 1307: 191-200). Bovine retina VLC-PUFAs were extracted and employed as VLC-PUFA standards to establish retention times because commercial standards are not available, and identification of each VLC-PUFA in retinal samples was achieved as described in prior work (Liu, et al. (2010) J. Lipid Res. 51: 3217-3229).

### i. Statistical Analyses

Statistical analyses were performed using analysis of variance (ANOVA), linear regressions, chi square tests, and t-tests on Prism software (GraphPad Software Inc., La Jolla, Calif., USA). Data are represented as the mean±SD. Significance is indicated by P value measurements, with P<0.05 considered significant.

2. Chemical Synthesis of VLC-PUFAS

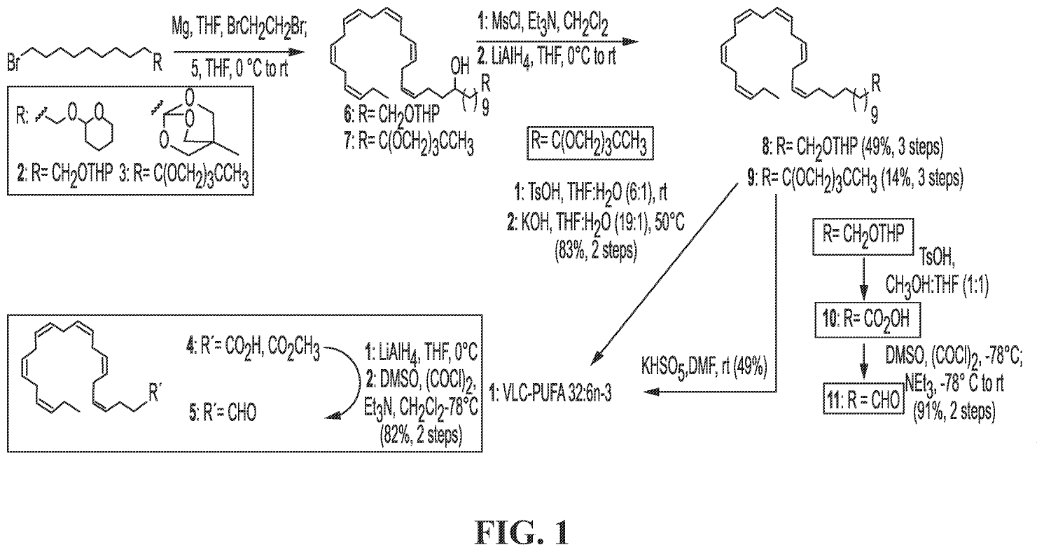

VLC-PUFAs are commercially available only in very small quantities, and published syntheses (Maharvi, et al. (2010) Tetrahedron Lett. 51: 6424-6428) had problematic coupling reactions, reductions, used toxic metals, and were judged by us not to be amenable to scale-up. The approach disclosed here to 32:6 n-3 (1) features the addition of Grignard reagents from either orthoester 3 or its acetal analog 2 to aldehyde 5 (FIG. 1). Manipulation of the coupled products 7 or 6, respectively, as illustrated allowed access to 1. The use of 3 as a precursor resulted in 6% overall yield of 1, while the use of 2 was much more productive and scalable, producing 1 in 29% overall yield. This newly reported approach was developed with the intention that synthetic VLC-PUFAs may eventually be used therapeutically in humans, so toxic materials were avoided. This synthesis also has the advantage of being amenable to scale-up and can be readily modified to synthesize other n-3 and n-6 VLC-PUFA family members and isotopically labeled versions.

3. The Influence of VLC-PUFAs on Membrane Structure and Dynamics

After developing an efficient synthetic route to producing VLC-PUFAs, their previously unexplored biophysical properties were studied, including their impact on membrane packing and compressibility as well their influence on membrane dynamics, particularly lipid flip-flop. VLC-PUFAs are present in retinal membranes at very low concentrations (<2%), but like cholesterol, such molecules may change membrane properties even at small mole percentages. The changes imparted by VLC-PUFAs on lipid packing and the compression moduli of model membranes were evaluated from π-A isotherms of 32:6 n-3 in 1,2-distearoyl-sn-3-glycero-phosphocholine (DSPC) lipid monolayers (FIG. 2A). In order to compare the attractive or repulsive forces between VLC-PUFA and DSPC lipid monolayers, the measured mean molecular areas (MMAs) were compared to the calculated MMAs of an ideal mixture of VLC-PUFA and DSPC. The presence of 0.1 mol %, 1 mol %, and 10 mol % VLC-PUFA shifted the π-A isotherm to higher MMAs of the lipids comprising the membrane, as shown in FIG. 2A.

Referring to FIG. 2A, π-A isotherms of VLC-PUFA at 0 mol % (solid, dark blue), 0.1% (solid, orange), 1% (solid, light-blue), 10% (dash, light-blue), and pure VLC-PUFA (solid, red). All isotherms were collected over a PBS pH 7.4 subphase at 22° C., and represent the averages of three experiments.

The influence of VLC-PUFA on lipid dynamics, namely lipid translocation or “flip-flop” was investigated by sum-frequency vibrational spectroscopy (SFVS). A comparison of the flip-flop rates of pure DSPC bilayers and 0.1 mol % VLC-PUFA in DSPC at 46° C. is presented in FIG. 2B. At this temperature, the half-life of flip-flop in the absence of VLC-PUFA was 120±2 minutes, which is in good agreement with the previously determined half-life of DSPC as 124±1 minutes (Allhusen, et al. (2016) J. Phys. Chem. B 120: 3157-3168). The incorporation of 0.1 mol % VLC-PUFA significantly increased the DSPC flip-flop rate with a calculated half-life of 24.3±0.2 minutes at 45.8±0.3° C., increasing the DSPC flip-flop rate by 4-fold. This apparent increase in the rate of DSPC flip-flop in the presence of VLC-PUFA is correlated to changes in membrane packing, which also correlated to previous findings that phospholipids with higher MMAs flip-flop faster (Anglin, et al. (2010) J. Phys. Chem. B 114: 1903-1914; Anglin and Conboy (2008) Biophys. J. 95: 186-193). Without wishing to be bound by theory, these results demonstrate that 0.1 mol % of VLC-PUFA induces a dramatic change in lipid membrane structure and dynamics and are the first direct measurements of the impact of VLC-PUFA on the physical properties of model membranes at a molecular level. Relatively low levels of VLC-PUFAs (0.1 mol %) in DSPC membranes result in high elasticity that makes the membranes defensive against external forces. VLC-PUFA also improves lipid translocation, which makes them noteworthy in retinal membranes where there is high influx of retinoids. This enhancement of flip-flop by VLC-PUFAs provides an alternative non-enzymatic mechanism for retinoid translocation in photoreceptors in addition to translocation mediated by the ABCA4 enzyme (Quazi and Molday (2013) J. Biol. Chem. 288: 34414-34426) and may explain why lipofuscin levels are high in the retinal pigment epithelium (RPE) when ELOVL4 is dysfunctional (Karan, et al. (2005) Proceedings of the National Academy of Sciences of the United States of America 102: 4164-4169).

Referring to FIG. 2B, a comparison of the measured DSPC flip-flop rates as a function of temperature for a pure DSPC (lower line) and 0.1 mol % VLC-PUFA (top line) membranes is shown. Error bars represent ±SD. Data for DSPC flip-flop in a neat bilayer were taken from Liu and Conboy (2005) Biophys. I. 89: 2522-2532.

4. Bioavailability and Absorption of VLC-PUFAs

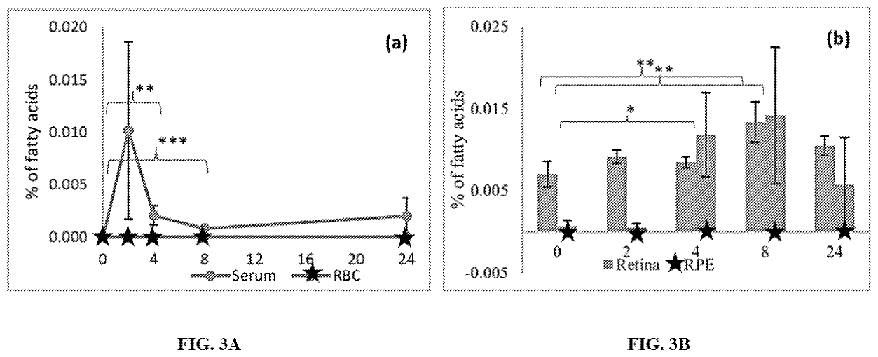

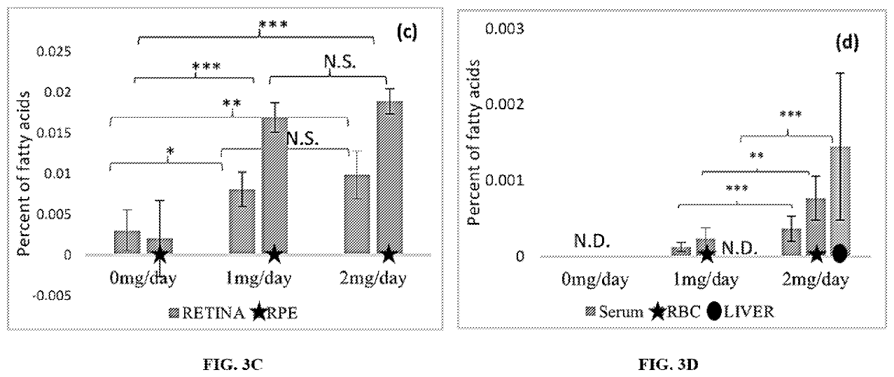

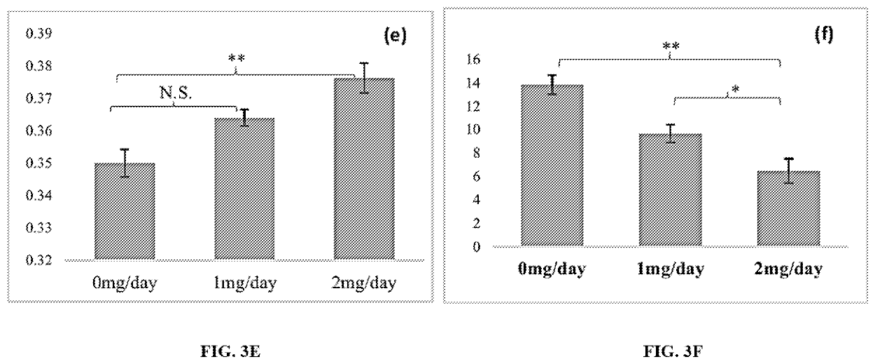

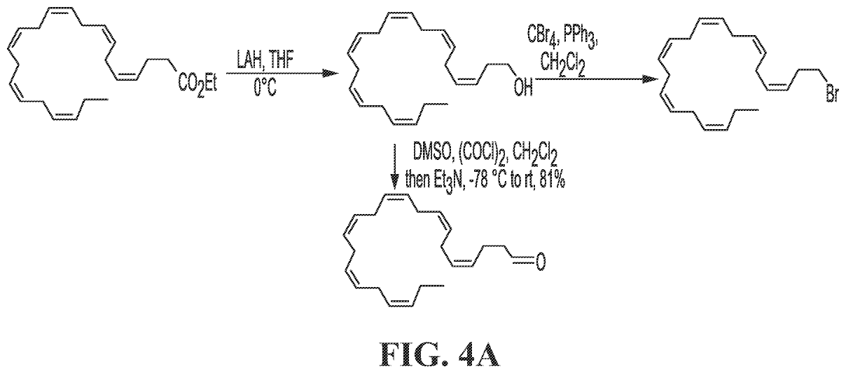

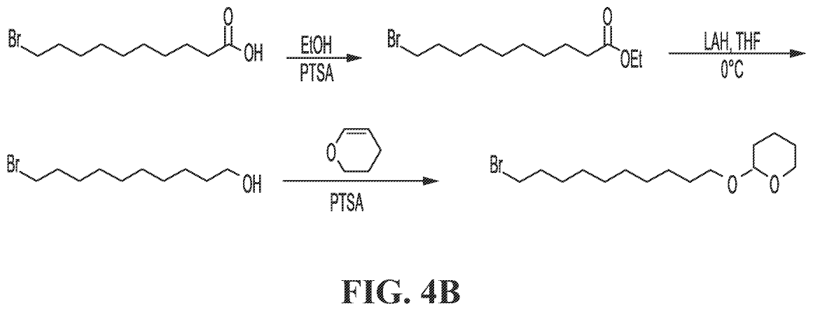

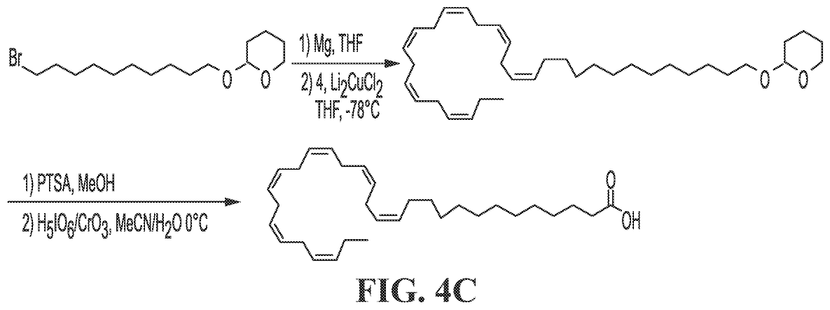

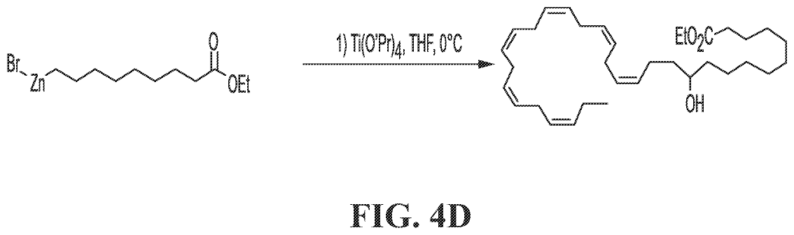

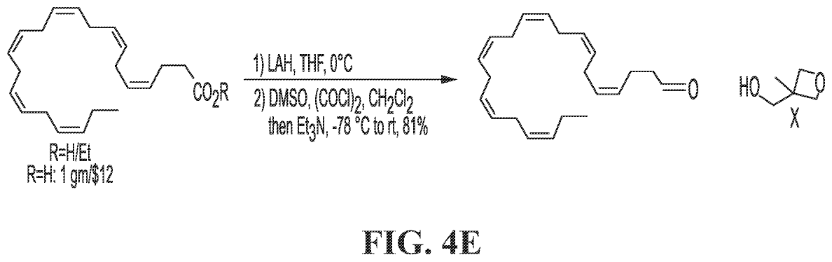

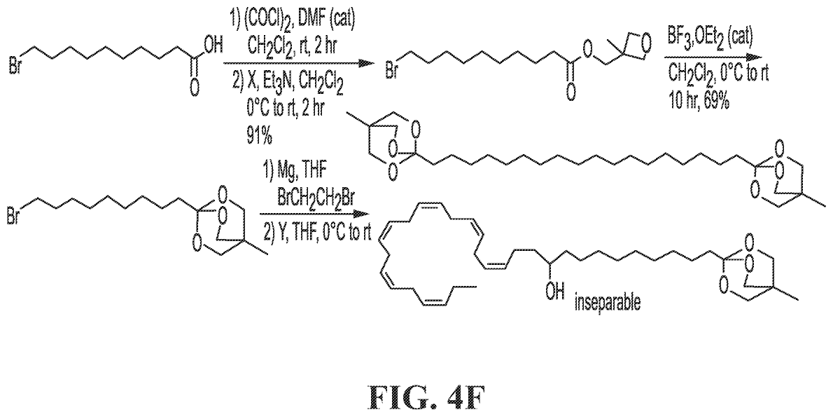

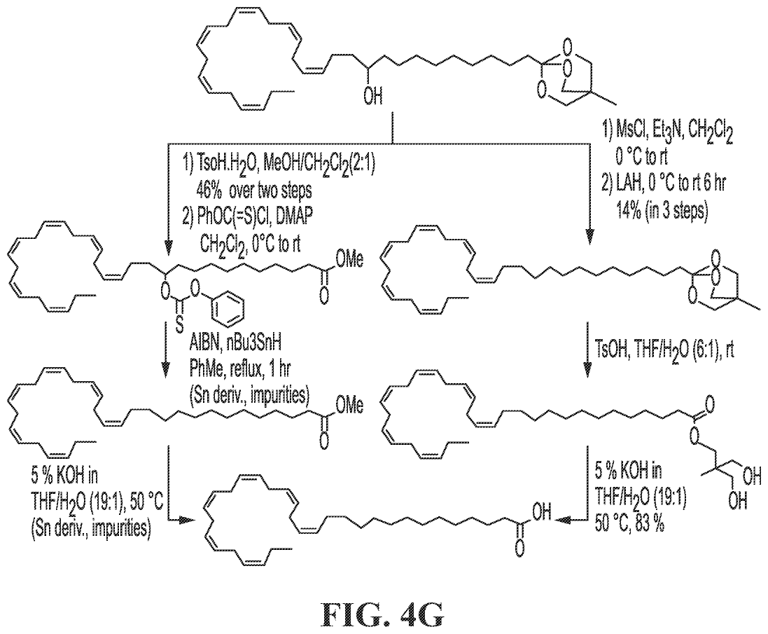

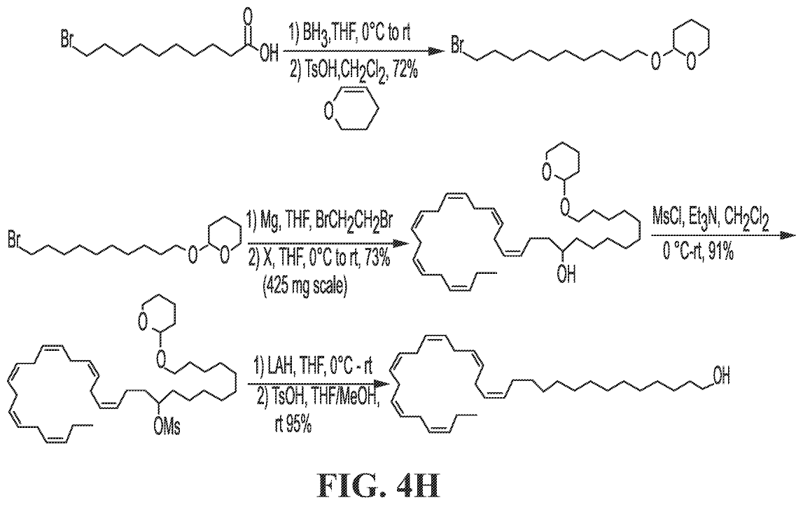

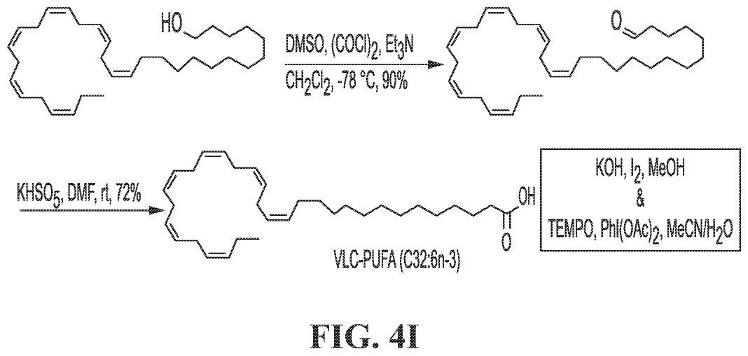

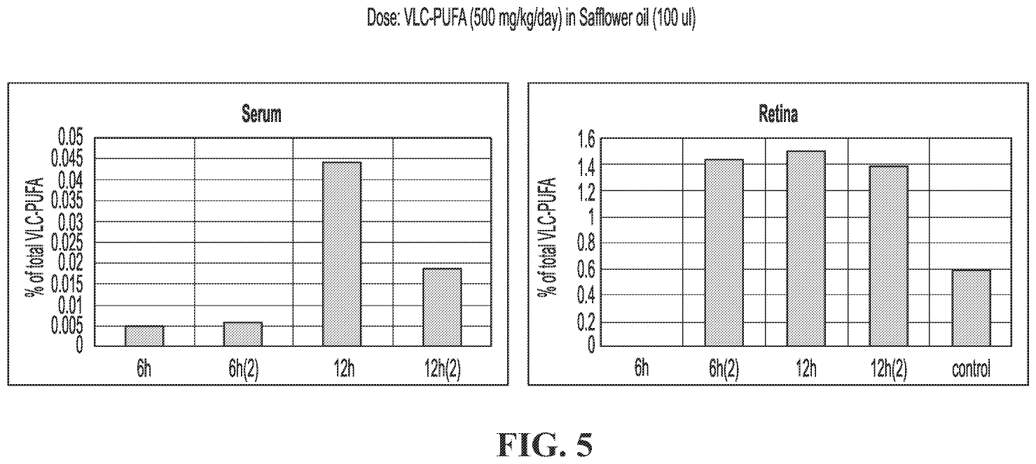

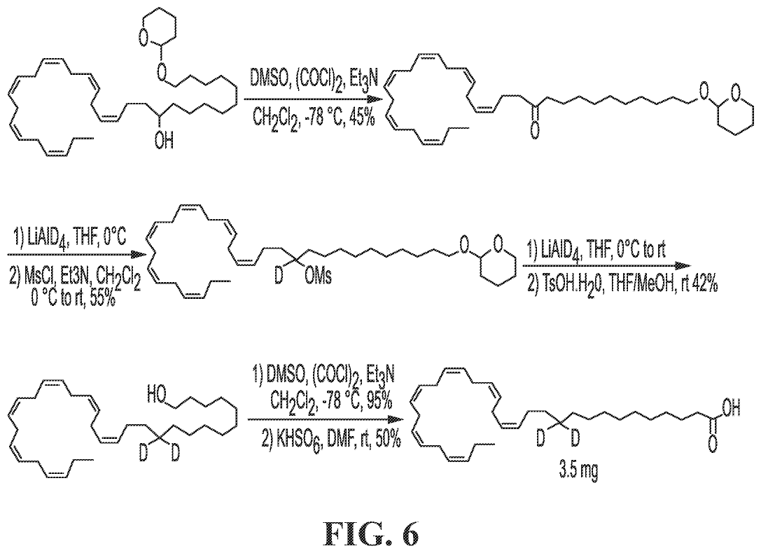

Next, the bioavailability of 32:6 n-3 was studied in mice after acute and chronic gavage feeding. In view of the physiological benefits of VLC-PUFAs and the dysfunction associated with low retinal levels of VLC-PUFAs, the feasibility of oral delivery of synthetic VLC-PUFA to mouse retinas was tested. Initially, mice received a single dose of 6 mg of 32:6 n-3 and were sacrificed 2, 4, 8, and 24 hr later (n=4 mice/time interval) to understand absorption kinetics. Vertebrate serum does not contain any VLC-PUFAs, but with single-dose gavage feeding of 32:6 n-3, serum VLC-PUFAs became detectable within 2 hr after oral administration and then reached a lower steady-state concentration over the next 24 hr (FIG. 3A). In retina and RPE, a significant increase in 32:6 n-3 was observed 4-8 hr after gavage (FIG. 3B). On the other hand, VLC-PUFAs remained undetectable in liver, brain, and red blood cell (RBC) membranes throughout the 24 hr-period.

For longer-term absorption studies, the mice were gavage-fed for 15 days with 0, 1, and 2 mg/day VLC-PUFA (n=6 mice/group). VLC-PUFA levels significantly increased by 2-fold in retina and by 7-fold in RPE in both VLC-PUFA groups in comparison to 0 mg/day controls (FIG. 3C). Serum and RBC VLC-PUFAs remained undetectable in the control mice and had significant increase in the VLC-PUFA-fed groups. VLC-PUFAs were detectable in the serum within hours, and with chronic feeding, they were also incorporated into RBC membranes at high and low doses. Liver had detectable levels of VLC-PUFAs only in the 2 mg/day group, while no VLC-PUFAs were detectable in brain (FIG. 3D). Earlier studies by Bazan's group have reported that VLC-PUFAs convert to elovanoids in vivo in RPE cells (Jun, et al. (2017) Sci. Rep. 7: 5279), but these analytical methods were not designed to detect the presence of these metabolites in eye tissues. Furthermore, toxic symptoms or behavioral changes were observed in treatment groups when compared to the control group. The visual acuity (cone function) and contrast sensitivity (rod function) of mice in the 2 mg/day-VLC-PUFA group improved significantly compared to controls (FIG. 3E and FIG. 3F). Thus, rapid and sustained increases of retinal and RPE VLC-PUFAs have been observed in response to acute and chronic supplementation that improved both rod and cone function in the mice, clearly demonstrating that orally administered VLC-PUFAs are bioavailable to ocular tissues and can exert positive effects on visual function.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composition comprising:
(a) a liposome;
(b) vitamin E; and
(c) a polyunsaturated fatty acid having a chain length of at least 24 carbon atoms, or a pharmaceutically acceptable salt thereof.

2. The composition of claim 1, wherein the liposome is prepared from Tween-4 detergent, cholesterol, safflower oil, and/or sodium taurocholate.

3. The composition of claim 1, wherein the liposome is a phosphatidyl choline.

4. (canceled)

5. The composition of claim 1, wherein the liposome is present in an amount of from about 0.5 wt % to about 1.5 wt %, based on the total weight of the composition.

6. (canceled)

7. The composition of claim 1, wherein vitamin E is present in an amount of from about 25 mg per 100 mL liposomes to about 50 mg per 100 mL liposomes.

8. The composition of claim 1, wherein vitamin E is present in an amount of from about 0.25 wt % to about 0.75 wt %, based on the total weight of the composition.

9. (canceled)

10. The composition of claim 1, wherein the polyunsaturated fatty acid is present in an amount of from about 5 wt % to about 50 wt %, based on the total weight of the composition.

11-12. (canceled)

13. The composition of claim 1, wherein the polyunsaturated fatty acid has a chain length of at least 28 carbon atoms.

14-16. (canceled)

17. The composition of claim 1, wherein the ratio of the liposome to vitamin E to the polyunsaturated fatty acid is of from about 1:0.5:40 (wt %) to about 1:0.5:10 (wt %).

18. The composition of claim 1, comprising a plurality of polyunsaturated fatty acids having a chain length of at least 24 carbon atoms.

19-20. (canceled)

21. The composition of claim 1, wherein the polyunsaturated fatty acid is an n-3 polyunsaturated fatty acid.

22-23. (canceled)

24. The composition of claim 1, wherein the polyunsaturated fatty acid has a chain length of from 24 carbon atoms to 36 carbon atoms, and is an n-3 polyunsaturated fatty acid.

25. (canceled)

26. The composition of claim 1, wherein the polyunsaturated fatty acid is an n6 polyunsaturated fatty acid.

27. (canceled)

28. The composition of claim 1, wherein the polyunsaturated fatty acid has a chain length of from 24 carbon atoms to 36 carbon atoms, and is an n6 polyunsaturated fatty acid.

29-30. (canceled)

31. The composition of claim 1, wherein the polyunsaturated fatty acid is fluorinated.

32. The composition of claim 1, wherein the polyunsaturated fatty acid is isotopically labeled.

33. (canceled)

34. The composition of claim 1, wherein the composition is formulated for oral administration, intraventricular injection, and/or ocular administration.

35-36. (canceled)

37. A method for treating an eye disorder in a subject in need thereof, the method comprising administering to the subject an effective amount of the composition of claim 1.

38-44. (canceled)

45. The method of claim 0, wherein the eye disorder is selected from Stargardt-3 (STGD3) disease, macular dystrophy, age-related macular degeneration (AMD), and diabetic retinopathy.

46. A method for supplementing a female subject's diet, the method comprising administering to the female subject an effective amount of the composition of claim 1, wherein the female subject is pregnant, desiring to become pregnant, or lactating.

47-135. (canceled)
